(19) United States
(12) Patent Application Publication
(10) Pub. No.: US 2025/0195498 A1
(43) Pub. Date: Jun. 19, 2025

(54) INHIBITOR OF CELLULAR PURINE NUCLEOTIDE SALVAGE PATHWAY ENZYME FOR USE IN THE TREATMENT AND/OR PREVENTION OF HELICOBACTER PYLORI INFECTION AND/OR DISEASE ASSOCIATED WITH SUCH INFECTION AND PHARMACEUTICAL COMPOSITION COMPRISING THE SAME

(71) Applicants: UNIWERSYTET WARSZAWSKI, Warszawa (PL); RUÐER BOŠKOVIC INSTITUTE, Zagreb (HR)

(72) Inventors: Maria Agnieszka Bzowska, Warszawa (PL); Marta Wojtys, Warszawa (PL); Katarzyna Jagusztyn-Krynicka, Warszawa (PL); Marta Narczyk, Warszawa (PL); Ivana Lešcic Ašler, Zagreb (HR); Zoran Štefanic, Zagreb (HR); Marija Luic, Zagreb (HR); Biserka Žinic, Zaprešic (HR); Weronika Maksymiuk, Warszawa (PL)

(73) Assignees: UNIWERSYTET WARSZAWSKI, Warszawa (PL); RUÐER BOŠKOVIC INSTITUTE, Zagreb (HR)

(21) Appl. No.: 18/847,221

(22) PCT Filed: Mar. 13, 2023
(86) PCT No.: PCT/PL2023/050014

§ 371 Date: Sep. 13, 2024

(30) Foreign Application Priority Data
Mar. 14, 2022 (PL) P.440651

(51) Int. Cl. A61K 31/4709; A61K 31/4164; A61K 31/4415; A61K 31/4439; A61K 31/675; A61K 31/7056; A61K 33/245; A61P 31/04

(52) U.S. Cl. CPC A61K 31/4709; A61K 31/4164; A61K 31/4415; A61K 31/4439; A61K 31/675; A61K 31/7056; A61K 33/245; A61P 31/04

(57) ABSTRACT

The present invention relates to an inhibitor of the Helicobacter pylori (H. pylori) cell purine nucleotide salvage pathway (reserve pathway) for use in the treatment of H. pylori infection and/or the treatment or prevention of a disease associated with this infection, such as gastritis and duodenitis, gastric and duodenal ulcers, as well as gastric cancer and MALT-type lymphoma, as well as pancreatic cancer or neurodegenerative diseases or disorders. The present invention also provides a pharmaceutical composition comprising such an inhibitor of the H. pylori cell purine nucleotide salvage pathway and at least one pharmaceutically acceptable excipient. The present invention further provides a pharmaceutical composition comprising such an inhibitor of the H. pylori cell purine nucleotide salvage pathway, at least one pharmaceutically acceptable excipient and at least one additional drug for the treatment of H. pylori infection, selected in particular from an antibiotic, a proton pump inhibitor and a bismuth salt.

The present invention relates to an inhibitor of an enzyme of the Helicobacter pylori cell purine nucleotide salvage pathway, preferably selected from purine nucleoside phosphorylase (PNP) and adenylosuccinate synthetase (AdSS), or a precursor thereof, for use in the treatment and/or prevention of Helicobacter pylori infection and/or a disease associated with Helicobacter pylori infection. The use of a single enzyme inhibitor of this pathway provides for an effective inhibition of the Helicobacter pylori growth, what makes it possible to use such inhibitor in the treatment and/or prevention of Helicobacter pylori infection and/or a disease associated with Helicobacter pylori infection. It is also possible to use for this purpose a combination of inhibitors of two enzymes of this pathway. Furthermore, such Helicobacter pylori cell purine nucleotide salvage pathway enzyme inhibitor, either alone or in a combination with an inhibitor of another enzyme belonging to this pathway, can be used in combination with other drugs currently used in the treatment of Helicobacter pylori infection, such as antibiotics, proton pump inhibitors and bismuth salts, i.e. in combination therapy. The present invention relates also to a pharmaceutical composition comprising such purine nucleotide salvage pathway enzyme inhibitor and a pharmaceutically acceptable excipient, optionally in combination with at least one other known drug used in the treatment of H. pylori infection.

Helicobacter pylori (H. pylori) bacterium, which was discovered over 30 years ago, and which colonizes the gastric wall, is a dangerous pathogen and cause of serious health problems worldwide (Makola et al., 2007). It is estimated that more than half of human population is infected with the bacterium, and in some countries, including Poland, and in certain age groups, the percentage of infected people reaches up to 80% (Dzierżanowska-Fangrat et al., 2005). Although in many cases infections are asymptomatic, the bacterium is considered one of the most dangerous human pathogens, as it is responsible for the development of many diseases, including gastric neoplasm. International Agency for Research on Cancer has recognized H. pylori as the class I carcinogen. H. pylori infection causes chronic inflammation of the stomach, which, if left untreated, can progress to gastric ulceration, duodenal ulceration, and eventually gastric cancer. In addition, H. pylori infection has been linked to mucosa-associated lymphoid tissue lymphoma (MALT) (Go, 2002), and even a pancreatic cancer (de Martel et al., 2008). According to recently published data, H. pylori infection has also been linked to neurodegenerative disorders (Malfertheiner et al., 2012). Diseases associated with H. pylori infection therefore include inter alia gastritis and duodenitis, gastric and duodenal ulcers, as well as gastric cancer and MALT lymphoma, as well as pancreatic cancer or neurodegenerative diseases or disorders. Combating H. pylori infection at an early stage will therefore help prevent its progression, leading to the development of diseases associated with the infection indicated above.

Currently used therapies for combating H. pylori infection are ineffective in case of over 20% of patients. A vaccine against H. pylori infection is not available either. Therapies that have been used so far in the treatment of H. pylori infection are most often based on the use of the several therapeutic agents. For example, the so-called triple or sequential therapy is used, which is based, for example, on the use of an inhibitor of an enzyme, so called proton pomp, in combination with two antibiotics: amoxicillin and clarithromycin for 7 to 14 days. Another exemplary therapy used in the treatment of H. pylori infection is the so-called quadruple therapy, based on a combination of four therapeutic agents, including antibiotics, proton pump inhibitor and bismuth salt (Huang et al., 2017). Unfortunately, these therapies are not always effective, not least because some patients are not susceptible to treatment and a number of H. pylori strains are resistant to antibiotics (Ruggiero, 2012). The number of resistant strains is expected to rise as the use of antibiotics increases, so intensive research is still being conducted in this field to improve the effectiveness of treatment by, among other things, overcoming antibiotic resistance. New therapeutic agents are also being intensively sought to combat H. pylori using molecular mechanisms of action other than those used to date, that is, purposing new targets for H. pylori infection therapy. Such agents may allow a reduction in the use of currently applied antibiotics and thus help reduce the emergence of antibiotic-resistant strains. Indeed, antibiotic resistance is a major challenge in the treatment of H. pylori infection and prevention of diseases associated therewith, because this bacterium has a number of pumps that actively remove undesirable substances, including antibiotics, which is one of the important mechanisms of drug resistance in many strains of this bacterium (Raj et al., 2021).

The life of all organisms depends on the synthesis of nucleic acid molecules, DNA and RNA. Purines, purine nucleosides and nucleotides are essential building blocks for the synthesis of these acids. A number of organisms, including H. pylori (but not the host organism, i.e. human), cannot synthesize de novo purines, purine nucleosides and nucleotides. They can only be derived from recycling metabolic waste, specifically from the purine salvage metabolic pathway, also called the purine nucleotide backup or reserve pathway or route.

Inhibition of this pathway, using an inhibitor of an enzyme belonging to this pathway, may therefore represent a promising therapeutic approach that offers the possibility of inhibiting the growth of microorganisms, such as H. pylori, which cannot synthesize de novo purines, as well as their nucleosides and nucleotides.

One of the cellular purine nucleotide salvage pathway enzymes is purine nucleoside phosphorylase (PNP; E.C. 2.4.2.1). This enzyme of purine nucleotide metabolism occurs in most living organisms and performs a following reaction:

$$β - purine nucleoside + orthophosphate ↔ purine base + α - D - pentose - 1 - phosphate$$

PNP from H. pylori is characterized by a broad spectrum of specificity toward substrates and inhibitors, in contrast to the human PNP. Furthermore, it has turned out that a deletion of a potential PNP gene, deoD, has resulted in an inability of H. pylori to grow on a purine nucleoside medium and one of purine bases, adenine (Liechti i Goldberg, 2012).

Inhibitors recently identified and described in the literature as potent in vitro inhibitors of the PNP enzyme from Plasmodium falciparum is mefloquine (in the hydrochloride form) and quinine (Dziekan et al., 2019). P. falciparum, similarly as H. pylori, does not synthetize de novo purines, purine nucleosides and nucleotides. Quinine was isolated for the first time in 1820 from the bark of the quinine tree Cinchona L. growing in South America in the Andes, while its derivative, mefloquine, was synthetized in 1984. Both compounds have found application in the treatment of malaria caused by protozoa from the Plasmodium genus. So far, however, the potential use of such PNP enzyme inhibitors for the treatment and/or prevention of H. pylori infection and diseases associated therewith has not been described.

Another promising example of PNP enzyme inhibitors are immucillins, which mimic the transition state of the reaction catalysed by the PNP enzyme. Immucillins are substituted 9-deazapurines, comprising, for example, immucillin H and immucillin A. For example, DADMe-Immucillin G is known to show clinical potential for treating Plasmodium falciparum-induced malaria (Ducati et al., 2013). On the other hand, immucillin A (“galidesivir”) has antiviral properties and immucillin H (“forodesine”) has anti-leukemic properties (Schramm, 2018). So far, however, the potential use of PNP enzyme inhibitors from the immucillin group for the treatment and/or prevention of H. pylori infection and diseases associated therewith has not been described.

So far, therefore, the possibility of use of PNP enzyme inhibitors as a new therapeutic approach for the treatment of H. pylori infection has not been explored, more specifically the use of H. pylori PNP inhibitors, which at the same time do not inhibit activity of human PNP, as selective drugs for the prevention and/or treatment of H. pylori infection and/or diseases associated therewith, alone or in combination with other compounds, including those currently used to treat H. pylori infection and/or diseases associated therewith. Another enzyme of the cellular purine nucleotide salvage pathway is adenylosuccinate synthetase (AdSS, or IMP-L-aspartate ligase, EC 6.3.4.4.). This enzyme catalyses the following reaction:

$$IMP + asparate + GTP ↔ ( in the presence of {Mg}^{2 +} ) adenylosuccinate + GDP + {P}_{i}$$

AdSS from H. pylori is a protein having 411 amino acids in length showing 44.9% sequence identity to AdSS from E. coli. However, there are differences between the structure of AdSS from bacteria and other organisms, including vertebrates (such as mice). Therefore, for effective inhibition of this enzyme in bacterial cells, inhibitors specific for AdSS from bacteria are required, in particular H. pylori. Liechti i Goldberg (2012) have found that the enzyme is crucial for growth of H. pylori bacteria, because a mutant lacking this enzyme is capable of growing on medium supplemented only with adenine or adenosine, while its growth is significantly inhibited in nutrient-rich medium. However, so far the possibility of using inhibitors of this enzyme in the treatment of H. pylori infection and diseases associated therewith has not been investigated, more specifically, the possibility of using inhibitors of the AdSS from H. pylori, which simultaneously does not inhibit the activity of human AdSS, as selective drugs for the treatment of H. pylori infection and/or diseases associated therewith, either alone or in combination with other compounds, currently used in the treatment of H. pylori infection.

Known inhibitors of the AdSS enzyme are, for example, hadacidin and hydantocidin 5′-phosphate (HMP). However, hadacidin does not penetrate the interior of the H. pylori or is effectively excreted by these bacteria and thus does not inhibit the proliferation of bacteria (Wojtyś et al., 2021). This is a fairly typical problem of many drug candidates, that they do not penetrate well enough into the cells where the enzyme to be inhibited is located. This is especially true in the case of H. pylori, because, as mentioned above, the bacterium has a number of mechanisms to actively remove undesirable substances, including antibiotics. As such, hydantocidin from various sources is a very weak inhibitor of the AdSS enzyme. However, AdSS is inhibited by its phosphorylated form, hydantocidin 5′-phosphate, as well as by the so-called bisubstrate inhibitors, i.e. hybrids in which two inhibitors have been combined, each interacting with a different part of the enzyme's active site. In particular, very potent inhibitors of this type are hybrids of hadacidin and hydantocidin 5′-phosphate (HMP) described by Hanessian et al. (1999). Moreover, Dong and Fromm (1990) have shown that pyridoxal 5′-phosphate (PLP) leads to almost complete inactivation of AdSS from E. coli. Inhibitors of AdSS from H. pylori have not yet been studied, and their potential use in the treatment and/or prevention of H. pylori infection and/or diseases associated therewith has not been studied.

As described above, because currently used therapies for H. pylori infection and diseases associated therewith are often ineffective, and because of the ever-increasing problem of drug resistance, there is a great need for new agents for use in the treatment of H. pylori infection that are effective not only in treating H. pylori infection, but also in the prevention and/or treatment of diseases associated with H. pylori infection. There is also a need for new agents effective for the treatment of H. pylori infection that could be used in combination with currently used drugs for the treatment of H. pylori infection to increase the effectiveness of the treatment, especially for many antibiotic-resistant strains of H. pylori. There is also a need for new agents effective for the treatment of H. pylori infection, which would allow to increase the effectiveness of the treatment of H. pylori infection, to reduce its duration and to reduce the use of antibiotics during treatment, to reduce antibiotic resistance and/or reduce the formation of antibiotic-resistant strains of H. pylori.

Thus, the object of the present invention is to provide new agents effective in treating H. pylori infection, that would be effective not only for the treatment of this infection, but also for the prevention and treatment of diseases associated with H. pylori infection, such as gastritis, duodenitis, gastric ulcer, duodenal ulcer, gastric cancer, MALT-type lymphoma, pancreatic cancer and neurodegenerative disorders. The objective of the invention is also to provide new agents effective in treatment of H. pylori infection, including pharmaceutical compositions comprising the same, which could be used in combination with currently used drugs to treat H. pylori infection, particularly many antibiotic-resistant strains of H. pylori. The object of the invention is also to provide new agents effective for the treatment of H. pylori infection, including pharmaceutical compositions comprising the same, which would allow to increase the effectiveness of the treatment of H. pylori infection, to reduce its duration and to reduce the use of antibiotics during treatment, which would reduce antibiotic resistance and/or reduce the formation of antibiotic-resistant strains of H. pylori. These objects have been achieved by the inventions defined in the attached claims. Surprisingly, it was found according to the invention that inhibition of just one enzyme of the H. pylori cell purine nucleotide salvage pathway, preferably PNP or AdSS, effectively inhibits the growth of H. pylori. More specifically, contrary to reports from the state of the art, it has been found according to the invention that inhibition of only one enzyme from the H. pylori cell purine nucleotide salvage pathway effectively inhibits the growth of H. pylori, which is particularly surprising since current treatments for H. pylori infection are often based on multi-drug combination therapy. Unexpectedly, it has turned out that inhibitors of the H. pylori cell purine nucleotide salvage pathway enzyme can be used as agents that effectively inhibit the growth of H. pylori bacteria, allowing their effective use in the treatment of H. pylori infection and/or the prevention and treatment of diseases associated with this infection, including those caused by antibiotic-resistant strains of H. pylori. Indeed, it has been unexpectedly found that some of the inhibitors effectively inhibit the growth of H. pylori in bacterial culture at concentrations comparable to drugs currently used to combat this pathogen. Unexpectedly, it has also turned out that the inhibitors for use according to the invention can also be used in combination with known drugs used in the treatment of H. pylori infection, thus providing a new combination therapy that allows for reduced use of antibiotics, and reduced risk of antibiotic-resistant strains. Inhibitors of the cellular salvage pathway for use according to the invention can be effectively used in the treatment of H. pylori infection, as well as in inhibiting its progression, and thus in the prevention and/or treatment of diseases known in the field to be associated with this infection, such as gastritis, duodenitis, gastric ulcer, duodenal ulcer, gastric cancer, MALT lymphoma, pancreatic cancer and neurodegenerative disorder.

### SUMMARY OF THE INVENTION

The subject of the present invention is an inhibitor of the Helicobacter pylori (H. pylori) cell purine nucleotide salvage (reserve) pathway enzyme for use in the treatment of H. pylori infection and/or the treatment or prevention of a disease associated with this infection. According to the invention, inhibition of only one enzyme of the H. pylori cell purine nucleotide salvage pathway provides effective inhibition of the growth of H. pylori, allowing for the treatment of H. pylori infection and/or the prevention or treatment of the disease associated therewith.

Preferably, the inhibitor for use according to the invention is an inhibitor of enzyme selected from purine nucleoside phosphorylase (PNP) and adenylosuccinate synthase (AdSS), or a metabolic precursor thereof.

Preferably, the inhibitor for use according to the invention is a PNP enzyme inhibitor. More preferably, the PNP enzyme inhibitor for use according to the invention is selected from substituted purines (Formula I), substituted 9-deaza-purines (Formula II), substituted 8-aza-9-deaza-purines (Formula III), mefloquine and quinine.

Even more preferably, the PNP inhibitor for use according to the invention is a substituted 8-aza-9-deaza-purine, in particular 6-methylformycin A.

More preferably, the PNP enzyme inhibitor for use according to the invention is a substituted 9-deaza-purine, in particular an immucillin. Even more preferably, the immucillin herein is immucillin A (Formula IIa).

More preferably, the PNP enzyme inhibitor for use according to the invention is a substituted purine selected from 6-benzyloxy-2-chloropurine (6BnO-2Cl-Pu, Formula IV below), 6-benzylthio-2-chloropurine (6BnS-2Cl-Pu, Formula V below), 2-chloro-6-benzylthiopurine-2′-deoxy-9-ribofuranoside (6BnS-2Cl-Pu-9dr, Formula VI below), 6-benzylthiopurine (6BnS-Pu, Formula VII below) and 2,6-dichloropurine (2,6-diCl-Pu, Formula VIII below). Even more preferably, the inhibitor is 6BnS-2Cl-Pu, most preferably 6BnS-2Cl-Pu in crystalline form.

More preferably, the PNP enzyme inhibitor for use according to the invention is mefloquine or quinine.

More preferably, the inhibitor for use according to the invention is an AdSS enzyme inhibitor, or a metabolic precursor thereof.

More preferably, the AdSS enzyme inhibitor for use according to the invention is selected from among hydantocidin, hydantocidin 5′-phosphate, hybrid of hadacidin and hydantocidin 5′-phosphate, pyridoxal 5′-phosphate (vitamin B6), and a metabolic precursor of pyridoxal 5′-phosphate, preferably pyridoxal.

Even more preferably as the inhibitor of the AdSS enzyme for use according to the invention is pyridoxal 5′-phosphate (vitamin B6), or a metabolic precursor thereof, preferably pyridoxal.

More preferably, as the inhibitor for use according to the invention both the PNP enzyme inhibitor as described above and the AdSS enzyme inhibitor as described above are used. Such a combination of inhibitors of the H. pylori cell purine nucleotide salvage pathway enzymes can further inhibit said purine nucleotide salvage pathway and consequently cause stronger growth inhibition of the H. pylori bacteria.

More preferably, the inhibitor for use according to the invention as described above is used for the prevention and/or treatment of a disease associated with H. pylori infection, wherein said disease associated with H. pylori infection is a disease selected from the group consisting of gastritis, duodenitis, gastric ulcer, duodenal ulcer, gastric cancer, MALT-type lymphoma, pancreatic cancer and a neurodegenerative disorder.

Preferably, the inhibitor for use according to the invention as described above is used for the treatment of H. pylori infection and/or the prevention and/or treatment of a disease associated with H. pylori infection, wherein the H. pylori bacterium belongs to an antibiotic-resistant strain. More preferably such antibiotic-resistant strain of H. pylori is resistant to clarithromycin and/or metronidazole. Even more preferably, the inhibitor for use according to the invention for use in the case of infection with the strain of antibiotic-resistant H. pylori bacteria is a PNP enzyme inhibitor selected from quinine or mefloquine. Even more preferably, the inhibitor for use according to the invention for use in the case of infection with the antibiotic-resistant H. pylori strain is the inhibitor for use according to claim 17 or 18, wherein the enzyme inhibitor used is an AdSS enzyme inhibitor selected from pyridoxal 5′-phosphate (PLP) and its precursor, that is, pyridoxal, preferably PI-h.

Preferably, the inhibitor for use according to the invention as described above is used in combination with at least one antibiotic, selected in particular from the group consisting of metronidazole, clarithromycin, tetracycline, amoxicillin, levofloxacin, sitafloxacin and rifabutin, especially with at least two antibiotics from this group. Preferably, the inhibitor for use according to the invention as described above is used in combination with a proton pump inhibitor (PPI), selected in particular from the group consisting of omeprazole, lansoprazole, esomeprazole, rabeprazole and pantoprazole.

More preferably, the inhibitor for use according to the invention as described above is used in combination with bismuth salt, especially with bismuth citrate.

More preferably, as the inhibitor for use according to the invention the inhibitor of the PNP enzyme as described above is used in combination with an antibiotic. More preferably, the PNP enzyme inhibitor in such a case is 6BnS-2Cl-Pu, and the antibiotic is metronidazole.

More preferably, the inhibitor for use according to the invention as described above is additionally used in combination with an additional antibiotic and/or proton pump inhibitor and/or bismuth salt.

The subject of the present invention is furthermore a pharmaceutical composition comprising an inhibitor of the H. pylori cell purine nucleotide salvage pathway for use as defined above, and at least one pharmaceutically acceptable excipient.

Preferably, the pharmaceutical composition according to the invention as said inhibitor comprises an inhibitor of the PNP enzyme, in particular 6BnS-2Cl-Pu, immucillin A, quinine or mefloquine.

Preferably, the pharmaceutical composition according to the invention as said inhibitor comprises an inhibitor of the AdSS enzyme, in particular pyridoxal 5′-phosphate (vitamin B6), or its metabolic precursor, preferably pyridoxal.

The subject of the invention is also a pharmaceutical composition comprising an inhibitor for use as defined above, at least one pharmaceutically acceptable excipient, and at least one additional drug for the treatment of H. pylori infection, chosen in particular from among an antibiotic, a proton pump inhibitor and a bismuth salt. Such a pharmaceutical composition is particularly suitable for use in the combination treatment of H. pylori infection and/or a disease associated therewith.

Preferably, such pharmaceutical composition according to the invention as said inhibitor comprises a PNP enzyme inhibitor, especially 6BnS-2Cl-Pu, immucillin A, quinine or mefloquine, and at least one antibiotic, especially metronidazole.

Even more preferably, such a pharmaceutical composition according to the invention additionally comprises a proton pump inhibitor and/or a bismuth salt.

### DETAILED DESCRIPTION OF THE INVENTION

According to the invention, recombinant enzymes of the H. pylori cell purine nucleotide salvage pathway: purine nucleoside phosphorylase (PNP) and adenylosuccinate synthetase (AdSS), were obtained by genetic engineering methods. At the molecular level, their properties were experimentally characterized and analysed, in particular their three-dimensional structure with atomic resolution and interactions with ligands. In the case of AdSS, since crystal structures of human, mouse, E. coli and several other bacteria's AdSS were deposited in the Protein Data Bank but not AdSS from H. pylori, in order to obtain this structure, the purA gene (which encodes the AdSS enzyme in H. pylori) was isolated from the genomic DNA of H. pylori strain 26695, amplified by PCR and subcloned into an expression vector with plasmid vectors. The resulting construct was used for transformation and overexpression of the recombinant AdSS enzyme of H. pylori in the E. coli strain BL21 (DE3), after which a procedure for isolating the enzyme was developed.

Next, it was tested whether and to what extent the reaction catalysed by these enzymes could be inhibited using already known PNP and AdSS inhibitors from other organisms.

Unexpectedly, some of the known PNP and AdSS inhibitors from other organisms were found to inhibit PNP from H. pylori much more strongly. Subsequently, it was tested whether and to what extent the use of the above inhibitors, alone or in combination with each other and/or other known drugs used to treat H. pylori infection, inhibits the growth of H. pylori (in cell cultures). According to the invention, it has been unexpectedly found that inhibition of as little as one enzyme of the H. pylori cell purine nucleotide salvage pathway makes it possible to achieve effective inhibition of H. pylori growth, and ultimately effective treatment of H. pylori infection and/or prevention or treatment of the disease associated therewith.

A group of following compounds is known in the art: formycin A, formycin B and their analogues with additional or altered substituents in the ring (this ring is the 8-aza-9-deaza-purine ring), for example, 1-methylformycin A or 6-methylformycin B, which are good inhibitors of the PNP enzyme from E. coli bacteria (Bzowska et al., 1992). Both proteins, i.e. PNP from H. pylori and PNP from E. coli, have a very similar amino acid sequence (about 70% similarity, Tomb et al., 1997) and may have similar properties. Unexpectedly, however, it was found that although formycin A and formycin B inhibit PNP from E. coli with almost identical inhibition constant, about 5 μM, formycin B is a much stronger inhibitor of PNP from H. pylori than formycin A, as the inhibition constants for this enzyme are 14 μM for formycin A and 1 μM for formycin B (Wojtyś et al., 2021). This shows that despite the similarity of enzyme sequences, it is not obvious that a given compound that is an effective inhibitor of PNP from one organism, including one bacterium, will be an effective inhibitor of this enzyme in another organism, including another bacterium. In addition, formycin A is not an inhibitor of human PNP, and formycin B is a very weak inhibitor, and this is extremely important for achieving selective inhibition of PNP from H. pylori, because human PNP binds purine nucleosides quite weakly, while nucleosides with a 6-amino substituent (such as formycin A) does not bind practically at all (Bzowska et al., 2000). The inventors also showed that formycin A has an effect on slowing down the proliferation of H. pylori in culture, but applied only in high concentrations, and formycin B, despite being a better PNP inhibitor of H. pylori, practically does not inhibit the proliferation of the bacteria, but the reason for this, as the inventors showed, is the poor penetration of this compound into H. pylori cells. These results show that even very similar compounds, such as formycin B and formycin A, can penetrate the interior of H. pylori cells to very different degrees and consequently inhibit or not the proliferation of this bacterium. These preliminary tests also showed that in order to obtain a more potent inhibitor, simultaneously better penetrating the interior of H. pylori cells, it is necessary to possibly modify formycin A and B. Based on previous work with PNP from E. coli bacteria carried out by the inventors, it was found that 10-fold stronger inhibition could be obtained using a methylated derivative of formycin A-6-methylformycin A. Based on these studies, it is believed that it can also be an effective inhibitor of PNP from H. pylori and inhibit the growth of this bacterium.

According to the invention, the inventors have also demonstrated that compounds that are substituted purines are effective PNP inhibitors from H. pylori, which also effectively inhibit the growth of H. pylori. Particularly preferable inhibitors from this group of compounds, also because they do not inhibit the PNP of the host, i.e. human, proved to be 6-benzyloxy-2-chloropurine (6BnO-2Cl-Pu), 6-benzylthio-2-chloropurine (6BnS-2Cl-Pu), 2-chloro-6-benzylthiopurine-2′-deoxy-9-ribofuranoside (6BnS-2Cl-Pu-9dr), 2,6-dichloropurine (2,6-diCl-Pu), 6-benzylthiopurine (6BnS-Pu).

According to the invention it has been shown that another group of effective PNP inhibitors from H. pylori are substituted 9-deaza-purines, especially immucillins. Immucillins include, for example, immucillin H and immucillin A. Among the substituted 9-deaza-purines, it is the immucillins, especially immucillin A, that deserve special attention here, because, as mentioned above, the human PNP interacts practically not at all with nucleosides having the 6-amino substituent that immucillin A has. Thus, immucillins with 9-deazadenine as an aglycone, like immucillin A, are selective inhibitors of PNP from H. pylori. Unexpectedly, it has turned out that it is immucillin A that is both a very good inhibitor of PNP from H. pylori and also effectively inhibits the proliferation of this bacterium.

Research described in Dziekan et al., (2019) identified PNP as a target enzyme for mefloquine and quinine in Plasmodium falciparum. These inhibitors were also found according to the invention to be effective in inhibiting the proliferation of H. pylori. These inhibitors were also found to be effective according to the invention in inhibiting the proliferation of bacterial strains of H. pylori resistant to antibiotics currently used in therapies.

The results obtained according to the invention therefore indicate that the PNP inhibitors of H. pylori inhibit the growth of this bacterium, which allows their use in the treatment and/or prevention of H. pylori infection and/or diseases associated therewith, since inhibition of the progression of H. pylori infection will allow prevention of its development into a disease associated with H. pylori infection, such as gastritis, duodenitis, gastric ulcer, duodenal ulcer, and gastric cancer and MALT lymphoma, as well as pancreatic cancer or neurodegenerative diseases or disorders, among others.

Inhibitors of the AdSS enzyme of the purine nucleotide salvage pathway from various organisms are also known in the art. The AdSS inhibitor from E. coli, for example, are the following compounds: hadacidin and hydantocidin 5′-phosphate, as well as their hybrid (Hanessian et al., 1999). Studies conducted by the present inventors have confirmed that hadacidin also strongly inhibits AdSS from H. pylori. This means that the AdSS inhibitors described above can inhibit the growth of this bacterium and can be used to treat H. pylori infection and diseases associated therewith, similarly to PNP inhibitors as described above. Another inhibitor of the AdSS enzyme from E. coli is also known in the art, namely pyridoxal 5′-phosphate (vitamin B6) (Dong and Fromm, 1990). Studies conducted by the present inventors have confirmed that it is also a potent inhibitor of AdSS from H. pylori, and is also effective in inhibiting the proliferation of H. pylori. Studies conducted by the present inventors have also confirmed that inhibition of H. pylori proliferation is achieved when one of the metabolic precursors of vitamin B6 is administered, that is, compounds that, when phosphorylated, become inhibitors of AdSS, specifically pyridoxal, in the form of pyridoxal hydrochloride (PI-h), which presumably the bacteria phosphorylate to PLP, because, as demonstrated by the present inventors, PI-h alone, in principle, does not inhibit the reaction catalysed by AdSS from H. pylori. However, administration of other metabolic precursors of PLP, in particular pyridoxine (in the form of hydrochloride), a precursor compound often administered as vitamin B6, does not inhibit the proliferation of H. pylori.

As mentioned, according to the state of the art, a good inhibitor of AdSS from E. coli is hydantocidin 5′-phosphate (HMP), which in some cells is formed from hydantocidin through phosphorylation by the corresponding enzyme in the cell, while hydantocidin itself hardly inhibits the reaction catalysed by AdSS (Walters et al., 1997). According to the invention, it is believed that hydantocidin in H. pylori, like pyridoxal, will be phosphorylated to HMP and thus converted to an active form, inhibiting AdSS and consequently the proliferation of H. pylori.

The results obtained according to the invention therefore indicate that AdSS inhibitors from H. pylori inhibit the growth of this bacterium, and that as a potential drug both the AdSS inhibitor itself and its metabolic precursor converted to an AdSS inhibitor in cells by phosphorylation may be considered. This is important due to the fact that compounds comprising a phosphate group as negatively charged may poorly penetrate the interior of H. pylori cells. The possibility demonstrated by the present inventors to administer as a drug a precursor of an AdSS inhibitor, i.e. a compound without a phosphate group, is extremely important in the context of pharmacological applications.

The results obtained in accordance with the invention therefore indicate that inhibitors of the AdSS enzyme from H. pylori inhibit the growth of this bacterium, which allows their use in the treatment and/or prevention of H. pylori infection and/or a disease associated therewith, since inhibition of the progression of H. pylori infection will allow prevention of the infection development into a disease associated with H. pylori infection, such as gastritis, duodenitis, gastric ulcer, duodenal ulcer, and gastric cancer and MALT lymphoma, as well as pancreatic cancer or neurodegenerative diseases or disorders, among others.

According to the invention, it has been found that obtaining with the use of the described herein inhibitors of the H. pylori cell purine nucleotide salvage pathway (cellular purine nucleotide salvage pathway or cellular salvage pathway for short) the inhibition of already one enzyme of this cellular salvage pathway, such as PNP or AdSS, effectively inhibits the growth of H. pylori. However, due to the unique properties of this bacterium and the existence of very many mutants thereof as described herein, inhibition of only one enzyme of the cellular salvage pathway may not in every case be effective enough to achieve sufficient growth inhibition of a H. pylori strain, because according to the recently described enzymatic machinery of the H. pylori cell purine nucleotide salvage pathway, alternative combinations exist for key enzymatic reactions (Liechti and Goldberg, 2012). Therefore, according to the invention, it is believed that the simultaneous inhibition of two enzymes of the aforementioned H. pylori cell purine nucleotide salvage pathway, e.g., purine nucleoside phosphorylase (PNP) and adenylosuccinate synthetase (AdSS), will inhibit the aforementioned purine nucleotide salvage pathway to an even stronger degree, and consequently the growth of the H. pylori bacteria. Such combined inhibition of the two enzymes of the cellular salvage pathway may therefore provide combination therapy for H. pylori infection, which has an even stronger inhibitory effect on the growth of H. pylori. The use of such a combination of a PNP inhibitor and an AdSS inhibitor may also be preferably used for multi-drug combination therapy of H. pylori infection, e.g. caused by a (multi) antibiotic-resistant strain.

For the above-mentioned reasons, the present inventors have also conducted a study using an enzyme inhibitor of the H. pylori cell purine nucleotide salvage pathway in combination with known drugs used for the treatment of H. pylori infection. Specifically, based on a study using a representative PNP enzyme inhibitor (6BnS-2Cl-Pu) as the inhibitor of the enzyme of the pathway discussed herein, in combination with a representative antibiotic used to treat H. pylori infection (metronidazole), it was found that the two compounds exhibit a different mechanism of action, and that their combined use makes it possible to achieve effective inhibition of H. pylori growth. The data obtained suggest that the use of a PNP enzyme inhibitor in combination with an antibiotic makes it possible to achieve an additive antimicrobial effect, so such their use provides a combination therapy for the treatment of H. pylori infection, allowing more effective, including faster, treatment of this infection, with reduced use of the antibiotic, which in turn reduces the risk of emerging antibiotic-resistant strains. Due to their different molecular mechanism of action, the inhibitors for use according to the invention can also be used in combination with other known drugs used to treat H. pylori infection, such as proton pump inhibitors or bismuth salts.

In the context of the present invention, the term “cellular purine nucleotide salvage pathway enzyme” used herein interchangeably with the term “cellular salvage pathway enzyme” or “purine nucleotide salvage pathway enzyme” means any enzyme belonging to the purine nucleotide salvage pathway or route, otherwise known as the cellular purine recovery pathway or the purine nucleotide backup or reserve pathway, more specifically of the H. pylori cell. More preferably in the context of the present invention, the enzyme is, but not limited to, PNP and/or AdSS from H. pylori.

In the context of the present invention, the term “inhibitor of the cellular purine nucleotide salvage pathway enzyme,” used herein interchangeably with the term “cellular salvage pathway enzyme inhibitor” or “purine nucleotide salvage pathway enzyme inhibitor” means any chemical compound exerting an inhibitory effect on the activity of at least one enzyme of the cellular purine nucleotide salvage pathway, more specifically the H. pylori cell, or a metabolic precursor of such inhibitor, i.e., a compound which, after metabolic modification in the target cell, e.g., phosphorylation, hydrolysis, phosphorolysis or otherwise, exerts an inhibitory effect on the activity of at least one enzyme of the cellular salvage pathway. Such inhibitory effect on an enzyme of the H. pylori cell salvage pathway results in inhibition of the growth of H. pylori cells, thereby allowing eradication of this bacterium and consequently treatment of the infection caused by it, as well as prevention and/or treatment of a disease associated with H. pylori infection.

Preferably, in the context of the present invention, the cellular salvage pathway enzyme inhibitor is, but not limited to, a PNP enzyme inhibitor from H. pylori. Exemplary PNP inhibitors from H. pylori are herein, but not limited to, substituted purines, particularly purines substituted at the 2 position and/or 6 position of the ring, in particular: 6BnO-2Cl-Pu, 6BnS-2Cl-Pu, 2,6-diCl-Pu, 6BnS-Pu, and additionally at position 9 (Formula I below), in particular 6BnS-2Cl-Pu-9dr and 6-(p-nitro-BnS)-2Cl-Pu-9dr (Formula Ia below), substituted 9-deazapurines, especially 9-deazapurines substituted at position 2 and/or 6 of the ring, and additionally at position 9 (Formula II below), especially immucillin A (Formula IIa below), substituted 8-aza-9-deazapurines, especially 8-aza-9-deazapurines substituted in position 2 and/or 6 of the ring, and additionally in position 9 (Formula III below), in particular 6-methylformycin (note that the formycin ring is customarily numbered differently in the literature than the purine ring, which, using 6-methylformycin A as an example, is illustrated by Formula IIIa below).

Preferably, the PNP inhibitor from H. pylori is according to the invention “substituted purine, purine 9-deaza and purine 8-aza-9-deaza”. These terms refer to the following groups of compounds:

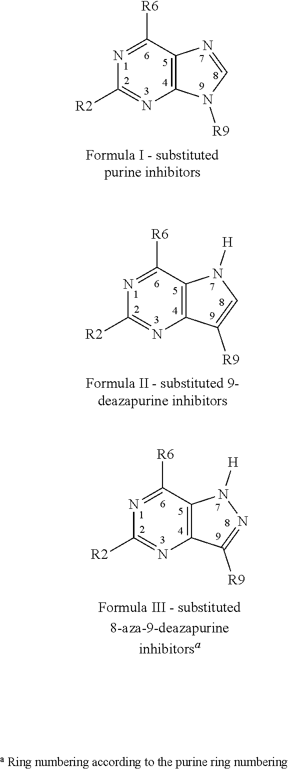

wherein R2, R6 and R9 are substituents at positions 2, 6 and 9 of purines, 9-deazapurines or 8-aza-9-deazapurines, preferably selected from, but not limited to, the following substituents: hydrogen, halogens, benzyloxy group, benzylthio group, and pentose and its modifications, such as carbasugars, iminosugars, acyclic chains and other cyclic and acyclic substituents. Exemplary representatives of each group are shown below:

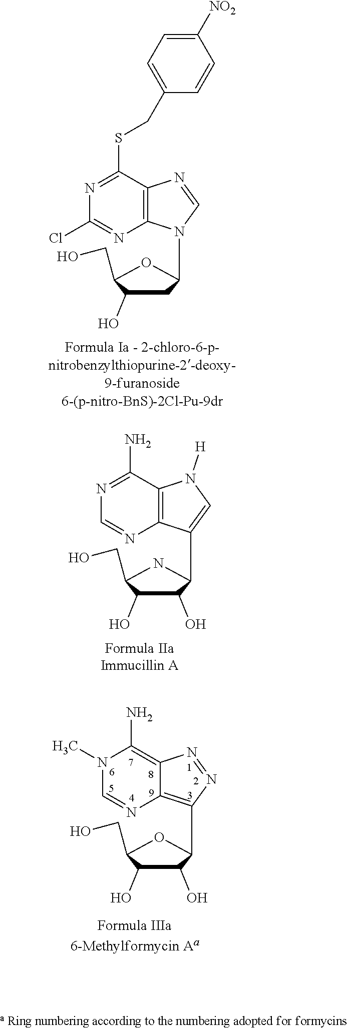

The PNP inhibitor from H. pylori for use in accordance with the present invention is also 6-methylformycin A.

The group of substituted 9-deaza-purines includes immucillins. In the context of the present invention, the term “immucillins” refers to compounds that are characterized by an NH moiety in a cyclic or acyclic substituent located at position 9 of the 9-deaza-purine ring. Due to the elevated pK of protonation, under neutral pH conditions the cyclic or acyclic substituent is bearing a positive charge, thus mimicking the transition state of the PNP-catalysed reaction in which the ribose (or 2′-deoxyribose) sugar ring is bearing a positive charge (Schramm, 2018). Immucillins include in particular the following compounds: immucillin A, immucillin H, immucillin G, which are analogues of PNP substrates, adenosine, inosine and guanosine with 9-deazapurine instead of purine and iminoribitol instead of ribose, as well as their counterparts DADME, DATME, SA and SerMe, wherein the substituent at position 9 of the 9-deazapurine ring has been modified (Ducati et al., 2013).

A particularly preferable PNP inhibitor from H. pylori for use according to the invention was found to be immucillin A (Formula IIa), as it inhibits both PNP from H. pylori and the proliferation of H. pylori, and as it has a 6-amino substituent in the 9-deazapurine ring, it will be a selective inhibitor of PNP from H. pylori, as it will not interact significantly with the PNP of the host, i.e., human.

Inhibitors of PNP from H. pylori for use according to the present invention are also 2-chloro-6-substituted purines, for example, such as the following:

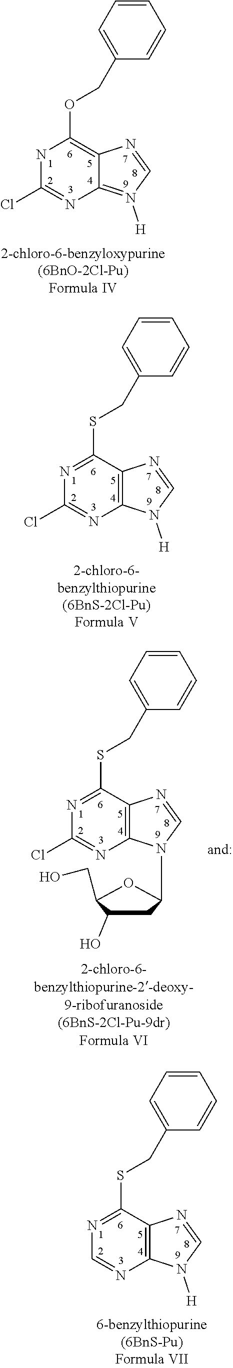

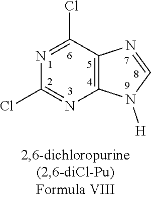 [SMILES: Clc1nc(Cl)c2nc[nH]c2n1]

Particularly preferable PNP inhibitors from H. pylori for use in accordance with the present invention are 6BnS-2Cl-Pu, 6BnO-2Cl-Pu, 6BnS-Pu and 2,6-diCl-Pu, as well as the metabolic precursor of 6BnS-2Cl-Pu, i.e. 6BnS-2Cl-Pu-9dr (which, as a PNP substrate, is converted to 6BnS-2Cl-Pu), wherein compounds with a benzylthio substituent at position 6 of the purine ring show the best inhibitory properties against H. pylori growth.

Particularly preferable PNP inhibitors of H. pylori for use according to the invention are mefloquine and quinine, especially in the case of the treatment of antibiotic-resistant H. pylori infection.

Preferably, in the context of the present invention, an inhibitor of the cellular purine nucleotide salvage pathway enzyme is, but not limited to, an inhibitor of the AdSS enzyme. Exemplary, preferable, inhibitors of AdSS from H. pylori herein are, but not limited to, hydantocidin (an analogue of the AdSS substrate-IMP, binding at a different site than hadacidin, but binding strongly only after phosphorylation at the 5′ ribose position), hydantocidin 5′-phosphate, hybrids of hadacidin and hydantocidin 5′-phosphate, pyridoxal 5′-phosphate (vitamin B6), and the metabolic precursor of pyridoxal 5′-phosphate, i.e. pyridoxal (like hadacidin binding strongly to AdSS only after phosphorylation). A particularly preferable AdSS inhibitor from H. pylori for use according to the invention is vitamin B6 and its metabolic precursor, pyridoxal. Inhibitors of the H. pylori cell nucleotide salvage pathway enzyme can be used according to the invention either alone, i.e., an inhibitor of one enzyme from the pathway, or in combination with each other, i.e., any PNP inhibitor together with any AdSS inhibitor, and/or additionally in combination with other drugs for the treatment of H. pylori infection, such as antibiotics, proton pump inhibitors and bismuth salts.

In the context of the present invention, the term “drug for the treatment of Helicobacter pylori (H. pylori) infection” refers to an antibiotic such as, but not limited to, metronidazole, tetracycline, amoxicillin, levofloxacin, sitafloxacin and rifabutin, wherein these antibiotics are often used in the treatment of H. pylori infection in combination (e.g., two antibiotics in combination); as well as to a proton pump inhibitor (PPI) such as, but not limited to, omeprazole, lansoprazole, esomeprazole, rabeprazole, pantoprazole, etc.; and to bismuth salts, including, but not limited to, bismuth citrate. According to the invention, these drugs can be used in combination with an inhibitor of at least one enzyme of the cellular salvage pathway, preferably an inhibitor of PNP and/or AdSS, as a combination therapy.

PNP from H. pylori was produced by the present inventors in E. coli cells, its kinetic and biochemical properties were characterized, and crystal forms of its complexes with 6BnO-2Cl-Pu, 6BnS-Pu, 2,6-diCl-Pu and other PNP inhibitors from H. pylori described in biochemical assays were obtained, and using these, the 3D structure of these complexes was determined with atomic resolution by X-ray diffraction.

It was shown that in each case the tested inhibitor of this enzyme binds to the active site of PNP from H. pylori (see FIG. 3, where 6BnS-2Cl-Pu binding to the active site of PNP is shown, as a representative example of PNP inhibitor binding to PNP from H. pylori). Details of each inhibitor's interaction with the enzyme's active site were also characterized. For each of the inhibitors tested, it was also found that the presence of the inhibitor results in a decrease in PNP activity from H. pylori and an arrest of the growth of the H. pylori bacterium in cell culture.

Inhibitors of the cellular salvage pathway enzyme for use according to the invention can be used in the form of a pharmaceutical composition comprising the same. In one aspect, the pharmaceutical composition according to the invention comprises as an active ingredient at least one inhibitor for use according to the invention as described above and at least one pharmaceutically acceptable excipient. In another aspect, the pharmaceutical composition comprises active ingredients and at least one pharmaceutically acceptable excipient, wherein one of the active ingredients is an inhibitor of the cellular salvage pathway for use according to the invention, another ingredient is at least one antibiotic, and yet another active ingredient is a proton pump inhibitor and/or a bismuth salt.

In one aspect, in the pharmaceutical compositions according to the invention, the inhibitor of the H. pylori cell purine nucleotide salvage pathway enzyme is any PNP enzyme inhibitor from H. pylori, in particular 6BnS-2Cl-Pu.

In a further aspect, in pharmaceutical compositions according to the invention, the inhibitor of the H. pylori cell purine nucleotide salvage pathway enzyme is any PNP enzyme inhibitor from H. pylori, in particular immucillin A, quinine or mefloquine.

In another aspect, in pharmaceutical compositions according to the invention, the inhibitor of the H. pylori cell purine nucleotide pathway enzyme is any AdSS inhibitor from H. pylori, in particular pyridoxal 5′-phosphate (vitamin B6) or pyridoxal.

In yet another aspect, in the pharmaceutical composition according to the invention, the active ingredient, in addition to an inhibitor of the cellular salvage pathway, is an antibiotic, in particular metronidazole. Such compositions may also comprise two antibiotics known in the field and used to treat H. pylori infection.

In still other aspects, pharmaceutical compositions according to the invention may further comprise a proton pump inhibitor and/or a bismuth salt.

Methods for producing pharmaceutical compositions comprising at least one active ingredient and at least one pharmaceutically acceptable excipient are known. Compositions according to the invention can be produced, for example, by mixing an active ingredient or active ingredients if, for example, a PNP enzyme inhibitor is used in combination with an AdSS enzyme inhibitor, or if a purine nucleotide salvage pathway enzyme inhibitor is used in combination with at least one other known drug for the treatment of H. pylori infection, with at least one excipient. Alternatively, such pharmaceutical compositions can also be produced, for example, by encapsulating the active ingredient(s) in such excipient(s) with the forming of, for example, a capsule, or other pharmaceutical form suitable for administration to an individual for the treatment of H. pylori infection, and/or the prevention or treatment of a disease associated therewith.

When the excipient used serves as a diluent, it can be a solid, semi-solid or liquid material that acts as a carrier or vehicle for the active ingredient. The pharmaceutical composition according to the invention can thus be, for example, in the form of a tablet, pill, powder, lozenge, elixir, suspension, emulsion, solution, syrup, aerosol, ointment containing, for example, up to 10% by weight of the active compound, soft and hard gelatin capsules, suppository, sterile injectable solution and sterile packaged powder.

Excipients suitable for use in pharmaceutical compositions are known. Some examples of these include, without limitation, lactose, dextrose, sucrose, sorbitol, mannitol, starch, acacia gum, calcium phosphate, alginates, tragacanth gum, gelatin, calcium silicate, microcrystalline cellulose, polyvinylpyrrolidone, water, syrup and methylcellulose. Such a composition may additionally contain: lubricating agents such as magnesium stearate, talc and mineral oil; wetting agents; emulsifying and suspending agents; preserving agents such as methyl benzoate and propylhydroxybenzoates; sweetening agents and flavouring agents. The pharmaceutical composition according to the invention may be in a form for rapid, sustained or delayed release of the active ingredient or ingredients. Methods of manufacturing such forms are known in the field.

The pharmaceutical compositions according to the invention can be formulated into a unit dosage form, wherein such unit dosage form is capable of containing from about 5 to about 500 mg of the active ingredient or active ingredients. The term “unit dosage form” refers to physically distinct units suitable for use as unit dosage forms in humans and other mammals, wherein each unit comprises a predetermined amount of the active ingredient or active ingredients calculated to provide the desired therapeutic effect, in this case inhibition of the growth of H. pylori, together with the corresponding pharmaceutically acceptable excipient.

The active ingredient can be effective over a wide dosage range and is usually administered in a pharmaceutically effective amount. It goes without saying that the actual amount of compound to be administered will be determined by the physician, according to the relevant circumstances of the case, including the disease being treated, the route of administration of the compound being used, the age and weight of the individual being treated, the severity of the symptoms, etc.

The present invention will now be illustrated in the following figures and examples, which, however, are not intended to limit in any way the scope of the invention as defined in the patent claims.

### BRIEF DESCRIPTION OF THE FIGURES

FIG. 1 shows the kinetic relationships observed for the catalysis of 7-methylguanosine by PNP from H. pylori illustrating the inhibition of the enzyme by immucillin A.

FIG. 2 shows the growth curves of H. pylori strain 26695 in cell culture in the presence of various concentrations of immucillin A. For clarity, only selected concentrations of immucillin A from the many tested are shown.

FIG. 3 shows a representative PNP inhibitor from H. pylori: 6BnS-2Cl-Pu (Formula V) bound to the active site of PNP from H. pylori. The grey spheres are water molecules present in the active site. Hydrogen bonds (up to 3 Å) between the ligands (6BnS-2Cl-Pu, Tris buffer molecule and phosphate ion) present in the active site and the enzyme are indicated by the dashed line. Analogous results were obtained with other PNP inhibitors (data not shown).

FIG. 4 shows the kinetic relationships observed for the catalysis of 7-methylguanosine by PNP from H. pylori illustrating the inhibition of the enzyme by the compounds 6BnO-2Cl-Pu (Formula IV), 6BnS-2Cl-Pu (Formula V), 6-BnS-Pu (Formula VII), 2,6-diClPu (Formula VIII) present at the concentrations described in the individual panels of this figure.

FIG. 5 shows the growth curves of H. pylori strain 26695 in cell culture in the presence of various concentrations of a representative PNP inhibitor from H. pylori: 6BnS-2Cl-Pu (Formula V) and 6-BnS-Pu (Formula VII). Both compounds were present either exclusively in dissolved form (right panels) or also in partially precipitated form (left panels). Kanamycin (25 μg/ml) was used as a positive control and water as a negative control. Shown is the optical density of cell cultures measured at 600 nm after 0, 4, 8 and 24 hours of incubation. The results shown are the average of three repetitions.

FIG. 6 shows examples of the colour change observed in the Christensen test in wells with H. pylori strain 26695 incubated in the presence of different concentrations of 6BnS-Pu (Formula VII). K⁺ indicates positive control (25 μg/ml kanamycin) and K indicates negative controls, with water (top) and BHI medium containing no H. pylori cells (bottom). Results shown are the average of three repetitions.

FIG. 7 shows the effects of a representative PNP inhibitor, 6BnS-2Cl-Pu, metronidazole (MTZ), and their combination, against replication of H. pylori strain 26695. Because the 6BnS-2Cl-Pu solution comprises DMSO (usually about 1% per 3 μg/ml of 6BnS-2Cl-Pu, the maximum used was 3.5%), the effect of MTZ solution containing 3.5% DMSO (bottom panel, second row) and DMSO alone, 3.5% and 2.3%, was also tested. The lower concentration of DMSO, 2.3%, has no effect on the growth of H. pylori cultures, while 26% inhibition is observed at 3.5% concentration. The percentage of inhibition of bacterial growth for each well is described. White circles with a cross in the centre indicate empty wells, K⁺ indicates positive control (kanamycin 25 μg/ml), and K indicates negative controls with water (top) and BHI medium containing no H. pylori cells (bottom). The results shown are the average of three repetitions.

FIG. 8 shows the growth curves of the reference H. pylori strain 26695 in the presence of different concentrations of the PNP inhibitors, mefloquine (a) and quinine (b).

FIG. 9 shows the growth curves of H. pylori strains resistant to clarithromycin (c), metronidazole (d) or both antibiotics simultaneously (a, b) in the presence of different concentrations of quinine, a PNP inhibitor. The results shown are the average of three repetitions.

FIG. 10 shows growth curves of H. pylori strains resistant to clarithromycin (c), metronidazole (d) or to both antibiotics simultaneously (a, b) in the presence of different concentrations of mefloquine (hydrochloride), a PNP inhibitor. The results shown are the average of three repetitions.

FIG. 11 shows the results of testing the inhibitory effect of a representative AdSS inhibitor from H. pylori, pyridoxal 5′-phosphate (PLP), present at various concentrations, on the inhibition of the reaction catalysed by AdSS from H. pylori at 25° C. in 20 mM Hepes/NaOH buffer pH 7.7, with saturating concentrations of AdSS substrates. One phase exponential decay was fitted to the data.

FIG. 12 shows the results of inactivation of AdSS from H. pylori by PLP as a function of incubation time. Top panel: graph of the dependence of inactivation of AdSS from H. pylori by PLP at different concentrations of the compound as a function of time. The enzyme (22 nM) was incubated at 25° C. in 20 mM Hepes/NaOH buffer pH 7.7, with PLP at different concentrations: 0.03 μM, 0.1 μM, 0.3 μM, 0.92 μM, 2.76 μM, 8.1 μM, and then the activity of AdSS was examined as described in Example 10. The activity observed at time 0 and in the absence of PLP is marked with two black circles (data from two different measurement days). One phase exponential decay was fitted to the relationships obtained for each concentration, in order to obtain the reaction rate constant k (rate constant) and the residual enzymatic activity when the system reached equilibrium, A_{∞}. Based on these parameters, the half-life of the inactivation activity process, t_{1/2}, was calculated. Lower panel: Dependence of the reaction rate constant, k(•), the residual enzymatic activity, A_{∞}(o), and the half-life of activity in the inactivation process, t_{1/2} (•) on inhibitor concentration, PLP.

FIG. 13 shows the results of a study to determine the minimum inhibitory concentration for the AdSS inhibitor from H. pylori, PLP, and its metabolic precursor, pyridoxal (PI-h): growth curves for the reference H. pylori strain 26695 (a, b), the H. pylori strain N6 (c, d) and the H. pylori strain P12 (e, f) in the presence of different concentrations of PLP and PI-h. The results shown are the average of three repetitions.

FIG. 14 shows the growth curves of four antibiotic-resistant H. pylori strains in cell culture in the presence of different concentrations of PLP (a), (c), (d) and (g) and PI-h (b), (e), (f) and (h). Kanamycin (25 μg/ml) was used as a positive control and water as a negative control. Shown is the optical density of cell cultures measured at 600 nm after 0, 4, 8 and 24 hours of incubation. The results shown are the average of three repetitions.

FIG. 15 shows pyridoxal 5′-phosphate and IMP bound to the active site of AdSS from H. pylori. The grey spheres are water molecules present in the active site. Hydrogen bonds (up to 3 Å) between ligands present in the active site and the enzyme are indicated by the dashed line. Pyridoxal 5′-phosphate occupies the site where the other AdSS substrate, that is, GTP, is bound, but unlike other ligands, in addition to hydrogen bonds it also forms a covalent bond with one of the active site's amino acids, Lys322.

### EXAMPLES

The invention is illustrated by the following examples, which are not however constituting a limitation thereof. Unless otherwise indicated, the following examples use known and/or commercially available equipment, methods, reaction conditions, reagents and kits, such as are commonly used in the field to which the present invention belongs and as are recommended by the manufacturers of the respective reagents and kits.

### Materials and Methods

### Materials

Guanosine (Guo), 2,6-dichloropurine (2,6-diCl-Pu, MW 189. 00 g/mol) was purchased from Sigma-Aldrich (Saint Louis, Missouri, USA), immucillin A was purchased from MedChemExpress (Sweden), magnesium chloride, sodium chloride and sodium dihydrogen phosphate, Tris were purchased from Roth (Karlsruhe, Germany), while Hepes preparations were from Roth (Karlsruhe, Germany) and Sigma-Aldrich (Saint Louis, Missouri, USA). NaOH (99% purity) was from POCh (Gliwice, Poland). 7-methylguanosine (m⁷Guo) was synthesized from guanosine by the method of Jones and Robins (1963) using methyl iodide. This ensured that the preparation was free of sulphate, which would have interfered with the results.

The PNP enzyme from Helicobacter pylori strain 26695 (HpPNP) was purified using affinity chromatography on a Sepharose-Formycin A column in a known manner (Narczyk et al., 2018).

Synthesis of known compounds: 6-benzyloxy-2-chloropurine (6BnO-2Cl-Pu, MW 260.68 g/mol) and 6-benzylthio-2-chloropurine (6BnS-2Cl-Pu, MW 276.74 g/mol) were synthesized by the known route as described in Bzowska et al., (1999). The synthesis of 6-benzylthiopurine (6BnS-Pu, MW 242.3 g/mol) was carried out by Dr. Biserka Žinić. The 2-chloro-6-benzylthiopurine-2′-deoxy-9-ribofuranoside (6BnS-2Cl-Pu-9dr, MW 393.87 g/mol) was donated by Prof. Zygmunt Kazimierczuk (Warsaw University of Life Sciences, Warsaw, Poland).

The AdSS enzyme from H. pylori strain 26695 was obtained in a known manner (Bubić et al., 2018). The substrates of the AdSS enzyme, i.e., GTP, IMP, aspartate and other chemicals used for testing AdSS activity, namely Hepes buffer, TCEP and MgCl₂, as well as PLP and its precursors were from Sigma-Aldrich (Saint Louis, Missouri, USA).

Spectrophotometric measurements, that is, collection of UV-VIS absorption spectra of enzyme, substrate and inhibitor solutions, as well as kinetic measurements of enzyme reaction rates, were performed using the Cary 100 dual-beam UV/VIS instrument with a Peltier thermostated cuvette holder (Varian: Agilent Technologies, Mulgrave, Vic., Australia). Spectral data (decimal molar extinction coefficients and differences in extinction coefficients between substrates and products) necessary for determining substrate, inhibitor concentrations and enzyme catalytic activity are given in the examples below.

Wild-type H. pylori strains 26695 and P12 (Schmitt and Haas, 1994) were obtained from ATCC (Manassas, Virginia, USA), while strain N6 was obtained from the Pasteur Institute,

Paris, France (Ferrero et al, 1992). Antibiotic-resistant strains M26, M91, M92, M93 were obtained from the Department of Microbiology, Faculty of Medicine, Wroclaw Medical University.

Bacterial culture reagents, specifically Fetal Bovine Serum (FBS), Helicobacter pylori Selective Supplement antibiotic kit and Brain Heart Infusion Broth (BHI medium) were purchased from Thermo Fisher Scientific (Waltham, Massachusetts, USA). Kanamycin, metronidazole, glycerol, methanol and dimethyl sulfoxide (DMSO) were from Sigma-Aldrich (Saint Louis, Missouri, USA).

Christensen's urea broth used in the urease viability test of H. pylori bacterium had the following composition: peptone-1 g/l, glucose-1 g/l, NaCl-5 g/l, Na₂HPO₄-1.2 g/l, KH₂PO₄-0.8 g/l, phenol red-0.004 g/l; 2% urea; pH 6.8. The reagents were from Sigma-Aldrich (Saint Louis, Missouri, USA).

The 24-well and 96-well microplates were purchased from Nest Scientific Biotechnology (New Jersey, USA). The microaerophilic atmosphere generator (anaerostat) for H. pylori culture, the Anoxomat Mark II, was from Mart Microbiology (Netherlands), while the microplate reader used to measure the optical density of H. pylori cultures was from Tecan (Switzerland). Sterile syringe filters with 0.22 μm pores were from Merck (Darmstadt, Germany).

Concentrated stock solutions of compounds for inhibition studies of the reaction catalysed by PNP and AdSS from H. pylori, and H. pylori growth inhibition in bacterial cultures were prepared as needed and depending on the solubility of the compound, in water, DMSO, methanol or BHI medium, and then diluted with buffer or BHI medium. In some cases, dilution to the planned final concentration was followed by precipitation of some of the compound. In such cases, either a partially precipitated preparation was used (which is indicated in the results presented), or the solution was filtered and the concentration of the remaining test compound in the supernatant was determined spectrophotometrically; the extinction coefficients used are given in the individual examples describing the testing of a particular inhibitor. Each time, the effect of the solvent used, at the final concentration present in a given experiment, on the rate of reaction catalysed by PNP or AdSS from H. pylori and on the growth rate of H. pylori in bacterial cultures was also checked.

### Example 1

Study of the use of selected PNP inhibitors, formycin A, formycin B, on the growth inhibition of H. pylori

Recombinantly produced PNP enzyme from H. pylori was purified to homogeneity by using an affinity column made by the present inventors to purify PNP from E. coli.

The specific activity of PNP is usually determined using inosine as a substrate, in an assay in which the phosphorolysis product, hypoxanthine, is oxidized to uric acid in a reaction catalysed by xanthine oxidase. However, in the inhibition assay of PNP from H. pylori strain 26695, 7-methylguanosine (m⁷Guo) and guanosine (Guo) were used as nucleoside substrates to avoid potential inhibition of xanthine oxidase by the test compounds. Phosphorolysis of these two substrates can be observed using a direct spectrophotometric method in a known manner. The spectral data for determining substrate concentration and enzymatic activity used in this study, are as follows: εₘₐₓ=13,650 M⁻¹ cm⁻¹ for Guo, Δε=−4,850 M⁻¹ cm⁻¹ at λ_{obs}=252 nm for Guo undergoing phosphorolysis to guanine (Bzowska et al., 1990) and εₘₐₓ=8,500 M⁻¹ cm⁻¹ for m⁷Guo (at pH 7.0), Δε=−4,600 M⁻¹ cm⁻¹ at λ_{obs}=258 nm for m⁷Guo undergoing phosphorolysis to 7-methylguanine (at pH 7.0) (Kulikowska et al., 1986).

Enzymatic activity was studied in a quartz cuvette with an optical path length of 1 cm or 0.5 cm. The absorbance of the reaction mixture, at the observation wavelength, never exceeded 1.2. The reaction volume was 1 ml or 1.4 ml in cuvettes with optical path lengths of 1 cm and 0.5 cm, respectively. The reaction mixture contained 50 mM Hepes/NaOH buffer, pH 7.0, 50 mM phosphate buffer, pH 7.0 (unless otherwise indicated), nucleoside substrate and inhibitor (with the latter not added to the reference/control reactions). Reactions were usually initiated by adding the enzyme to a cuvette containing the other reaction components. However, in cases where possible slow formation of an inhibitor-enzyme complex was checked, a second variant was also used involving incubation of the enzyme in a reaction mixture containing the inhibitor but lacking the substrate, 7-methylguanosine, and initiation of the reaction by adding this substrate.

In order to determine the inhibition constants and determine the molecular mechanism of inhibition, equations describing different mechanisms of inhibition were fitted to the obtained experimental data. GraphPad Prism 8 program was used. The model best describing the obtained data was selected based on the sum of squares of deviations using the F test for models with different number of parameters and the Akaike criterion (AIC) for models with the same number of parameters.

The following equations were used to describe competitive inhibition, characterized by the inhibition constant K_{ic}, [1], uncompetitive, characterized by the inhibition constant Kᵢᵤ, [2], non-competitive, characterized by the inhibition constant Kin, [3], and mixed, characterized by the inhibition constants Kᵢᵤ and K_{ic}, [4] (Segel, 1993):

$${v}_{o} ( {c}_{o} ) = \frac{{V}_{max} {c}_{o}}{{c}_{o} + ( 1 + \frac{[ I ]}{{K}_{ic}} ) {K}_{m}} \qquad [ 1 ]$$

$${v}_{o} ( {c}_{o} ) = \frac{{V}_{max} {c}_{o}}{( 1 + \frac{[ I ]}{{K}_{iu}} ) {c}_{o} + {K}_{m}} \qquad [ 2 ]$$

$${v}_{o} ( {c}_{o} ) = \frac{{V}_{max} {c}_{o} / ( 1 + \frac{[ I ]}{{K}_{in}} )}{{c}_{o} + {K}_{m}} \qquad [ 3 ]$$

$${v}_{o} ( {c}_{o} ) = \frac{{V}_{max} {c}_{o}}{( 1 + \frac{[ I ]}{{K}_{iu}} ) {c}_{o} + ( 1 + \frac{[ I ]}{{K}_{ic}} ) {K}_{m}} \qquad [ 4 ]$$

The last equation can also be represented in the following form [5], as used by GraphPad Prism software, where Kᵢᵤ=alpha K_{ic}:

$${v}_{o} ( {c}_{o} ) = \frac{{V}_{max} {c}_{o}}{( 1 + \frac{[ J ]}{{alphaK}_{ic}} ) {c}_{o} + ( 1 + \frac{[ I ]}{{K}_{ic}} ) {K}_{m}} \qquad [ 5 ]$$

Kinetic results obtained by the methods described above confirmed that the PNP enzyme from H. pylori is strongly inhibited by formycin A (FA) and formycin B (FB). Unexpectedly, however, FB was found to be a much more potent inhibitor of PNP from H. pylori than FA, with inhibition constants of 14 μM and 1 μM, respectively, although both compounds inhibit PNP from E. coli with almost identical inhibition constant, approx. 5 μM.

Then, the present inventors demonstrated, on a culture of H. pylori, using the method described in detail in Example 3, that formycin A (FA) actually inhibits the growth of H. pylori, but it is necessary to use a high concentration of FA (2.5 mM) to achieve a significant level of inhibition (50% of the growth observed without the inhibitor). In contrast, formycin B (FB), even at this high concentration, shows very weak inhibition of H. pylori proliferation. This result shows that PNP inhibitors have an effect on the growth rate of H. pylori in culture, and at the same time suggests that for some of such inhibitors, it may be necessary to apply a method to improve the efficiency of inhibitor penetration into the cells of H. pylori bacteria, which is a typical problem for a number of substances that are potential drugs.

This result also shows that a compound showing even very strong inhibition of a target enzyme from H. pylori, let alone a target enzyme from another organism, is not equivalent to inhibiting the proliferation of H. pylori bacteria. Thus, whether a given inhibitor, even one known and described in the literature as inhibiting PNP from an organism, will be a promising drug candidate to combat H. pylori infection is not obvious to the expert, and it is necessary to directly test the effect of that compound on the reaction catalysed by PNP from H. pylori and the effect of that compound on H. pylori cell cultures.

### Example 2

Study of the PNP Enzyme from H. pylori Inhibiting Properties by a Selected Immucillin

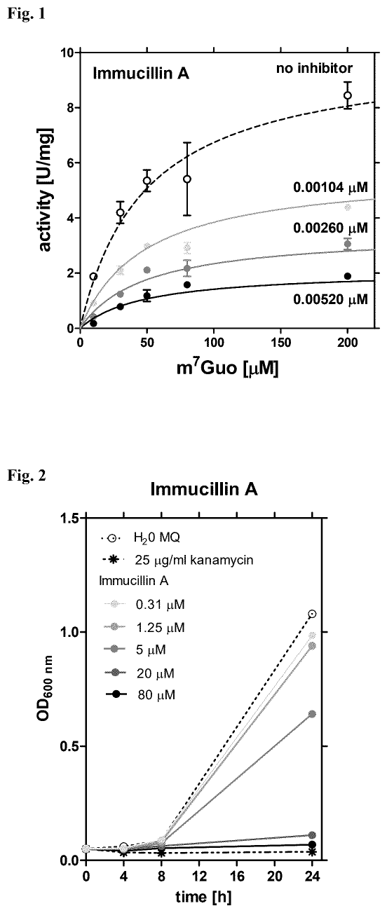

Immucillins, as transition state analogues of the phosphorolysis reaction catalysed by PNP, are the most potent known inhibitors of this enzyme. Immucillin A was chosen as the most promising one for potential pharmacological use in combating H. pylori infection because immucillin A has an amino substituent at position 6 of the 9-dazapurine ring, while PNP found in the human body requires a substituent at this position having electron-pairs-hydrogen bond acceptors, such as a substituent in the form of a keto group, to strongly bind the ligand. According to the invention, it is therefore believed that immucillin A will not interact with the host PNP, and thus allow selective inhibition of the PNP from H. pylori. Using the methods described above, the effect of immucillin A on the rate of the 7-methylguanosine (m⁷Guo) phosphorolysis reaction catalysed by PNP from H. pylori was examined (FIG. 1). Since it is known (Schramm, 2018) that immucillins form a strongly bound complex with the enzyme relatively slowly, reactions were initiated by adding the substrate, m⁷Guo, to a cuvette in which the enzyme was incubated with the inhibitor, as described in Example 1.

Based on the experimental data obtained, immucillin A was shown to be a potent inhibitor of PNP from H. pylori, inhibiting the enzyme according to equation [2], and thus in a non-competitive manner with respect to the nucleoside substrate, m⁷Guo, indicating, as expected, the slow formation of a stable complex of the enzyme with a strongly bound inhibitor (tight-binding complex). The determined inhibition constant is 0.0013±0.0002 μM.

### Example 3

Study of PNP Inhibition Effect by a Selected Immucillin on the Growth of H. pylori Cells

After confirming the very good inhibitory properties against PNP from H. pylori of compounds from the immucillin group for use according to the invention, the present inventors conducted tests to confirm whether these compounds inhibit the growth of H. pylori in cultures. The representative PNP inhibitor from H. pylori included in this study was immucillin A. The experiment was conducted using the growth curve method (two-fold serial dilutions in liquid medium). The tested H. pylori strains were cultured in Petri dishes with BHI solid medium supplemented with 10% FBS and 1% H. pylori Selective Supplement antibiotic kit, in anaerostats under microaerophilic conditions (5% 02, 10% CO₂, 85% N2) at 37° C. for 3 days. Liquid culture of H. pylori was conducted overnight with shaking at 37° C. under microaerophilic conditions in BHI medium supplemented with 10% FBS and 1% H. pylori Selective Supplement antibiotic kit. Subsequently, for the essential experiment of determining the minimum inhibitory concentration (MIC), double strengthened liquid BHI medium was prepared, that is, supplemented with 20% FBS and a 2% set of H. pylori Selective Supplement antibiotics.

Bacterial proliferation inhibition experiments were carried out in 24-deep-well 2 ml flat-bottomed plates at a solution volume per well of 0.5 ml. In a single well, double strengthened (relative to the concentration planned for testing) solutions of the test inhibitor and double strengthened liquid BHI medium inoculated with H. pylori (10⁴-10⁵ CFU/ml in the final volume in the well, corresponding to the optical density of the culture OD₆₀₀=0.05) were mixed together in a 1:1 ratio.

Kanamycin at a concentration of 25 μg/ml (Irie et al., 1997) was used as a positive control, while Mili-Q deionized water and BHI medium not inoculated with bacteria were used as negative controls. Plates were shaken at 37° C., under microaerophilic conditions. During the experiment, after 4, 8 and 24 hours of incubation, a culture sample was taken from each well and optical density measurements at 600 nm (OD₆₀₀) were taken in microplates using a microplate reader to determine MIC values. After 24 hours, the OD₆₀₀ of samples containing the negative control, i.e. with Mili-Q deionized water, was about 1.2-1.4, corresponding to a bacterial concentration of about 106 CFU/ml.

In addition, the method proposed by Knezevic et al. (2018) was used to check whether the observed OD₆₀₀ was from live bacterial cells. For this purpose, an equal volume of Christensen's double strength urea broth (that is, containing twice the concentration of all components) was added to the wells after incubation, and the plates were additionally incubated for 4 hours in an oxygen atmosphere at 37° C. During incubation, in wells with urease produced by live bacteria, urea is converted to ammonia and carbon dioxide, changing the pH of the solution and thus the colour of the phenol red indicator contained in the medium (from orange to purple).

The MIC is defined as the lowest concentration of a compound that inhibits visible bacterial growth (no colour change), compared to growth in the control group (presence of colour change), which corresponds to inhibition comparable to that observed in the positive control (with 25 μg/ml kanamycin), that is-inhibition of bacterial growth by 90% or more. All experiments were performed in triplicate.

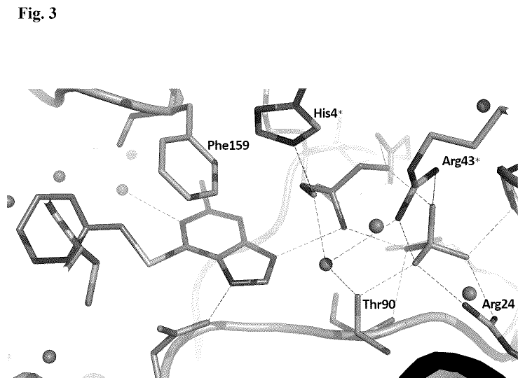

The study allowed to conclude that immucillin A, tested in a wide range of concentrations (FIG. 2), inhibits the growth of H. pylori, and the minimum inhibitory concentration MIC determined on the basis of these data is 80 μM.

### Example 4

Obtaining the Structure of the PNP Enzyme from H. pylori with the Representative PNP Inhibitors (6BnS-2Cl-Pu, 6BnO-2Cl-Pu, 6BnS-Pu, 2,6-diCl-Pu)

The crystal structures of complexes of the studied enzyme with selected PNP inhibitors were determined by X-ray diffraction and characterised.

Crystals of PNP complexes with the studied inhibitors were obtained by the hanging drop method under conditions described in Štefanić et al. (2017). The enzyme solution (9-10 mg/ml in 50 mM Tris/HCl buffer, pH 7.6) was mixed with a ligand solution (0.5-1.4 mM) and phosphate (0.5-0.6 mM) and, after 30 minutes of incubation, placed in crystallisation drops. The complex of PNP with 6BnS-2Cl-Pu was obtained by mixing the enzyme solution with a solution of its precursor, i.e. nucleoside, 6BnS-2Cl-Pu-9dr, which resulted in the formation of 6BnS-2Cl-Pu through a PNP-catalysed phosphorolysis reaction. In the case of 2,6-diClPu, ligand-free protein crystals were first obtained under conditions of 0.2 M imidazole pH 7.0 and PPG 400, and then inhibitor powder was added to the droplet containing the crystals. After 3 days of the complexation process, the crystals were frozen and diffraction data were collected.

Diffraction data for all crystals were collected using the XRD1 line of the Elettra synchrotron (Trieste, Italy), using a Dectris Pilatus 2M detector. Data were integrated using XDS software (Kabsch, 2010). All structures were solved by molecular replacement using the Molrep programme (Vagin and Teplyakov, 1997), using the H. pylori PNP structure (PDB code 5LU0, Narczyk et al., 2018) as a model. Models were refined using the phenix. refine procedure from the Phenix package (Liebschner, et al., 2019). The statistics for the collected data and the parameters of the obtained structures, after their refinement, are summarised in Table 1.

Coordinates of the determined structures and structural factors have been deposited in the Protein Data Bank database under the following PDB codes: 700Y for 6BnS-2Cl-Pu, 700Z for 6BnO-2Cl-Pu, 7OPA for 6BnS-Pu and 7OP9 for 2,6-diCl-Pu.

TABLE 1
Collected diffraction data and statistical analysis thereof, as well as parameters of the obtained structures.

| | 6BnS-2Cl- | | 6BnO-2Cl- | | | | 2,6-diCl- | |
|---|---|---|---|---|---|---|---|---|
| Structure | Pu | | Pu | | 6BnS-Pu | | Pu | |
| Resolution range | 35.9-1.9 | | 35.9-1.7 | | 46.35-2.0 | | 42.59-1.5 | |
| | (1.97-1.9)^{a} | | (1.76-1.7) | | (2.07-2.0) | | (1.55-1.5) | |
| Space group | P 2₁ 3 | | P 2₁ 3 | | P 2₁2₁2₁ | | P 1 | |
| Unit cell | | | | | | | | |
| a, b, c (Å) | 113.6; 113.6; 113.6 | | 113.7; 113.7; 113.7 | | 67.6; 139.0; 318.7 | | 93.4; 93.4; 95.4 | |
| α, β, γ (°) | 90. 90. 90 | | 90. 90. 90 | | 90. 90. 90 | | 81.9; 79.3; 60.1 | |
| Total number of reflections | 433440 | (40977) | 465820 | (44846) | 2504977 | (186198) | 1079039 | (98731) |
| Number of unique reflections | 38724 | (3843) | 54014 | (5331) | 202884 | (19800) | 419695 | (38205) |
| Multiplicity | 11.2 | (10.7) | 8.6 | (8.4) | 12.3 | (9.4) | 2.6 | (2.6) |
| Completness (%) | 99.97 | (99.97) | 99.93 | (99.61) | 99.83 | (98.78) | 94.95 | (86.85) |
| Mean I/σ(I) | 7.83 | (1.14) | 12.18 | (1.53) | 9.85 | (0.76) | 9.13 | (1.78) |
| Wilson B-factor | 20.88 | | 19.04 | | 36.70 | | 13.35 | |
| R_{merge} | 0.32 | (2.1) | 0.12 | (1.27) | 0.18 | (2.18) | 0.09 | (0.52) |
| Rₘₑₐₛ | 0.33 | (2.2) | 0.13 | (1.36) | 0.19 | (2.30) | 0.11 | (0.66) |
| Rₚᵢₘ | 0.10 | (0.69) | 0.04 | (0.46) | 0.05 | (0.73) | 0.07 | (0.39) |
| CC_{1/2} | 0.99 | (0.48) | 0.99 | (0.60) | 0.99 | (0.57) | 0.99 | (0.54) |
| CC* | 0.99 | (0.80) | 0.99 | (0.87) | 0.99 | (0.85) | 0.99 | (0.84) |
| Reflections used for refinement | 38720 | (3843) | 54008 | (5330) | 202855 | (19799) | 417539 | (38185) |
| Reflections used for calculation of | 1992 | (197) | 1989 | (197) | 1999 | (196) | 2013 | (187) |
| R_{free} | | | | | | | | |
| R_{work} | 0.16 | (0.26) | 0.16 | (0.28) | 0.23 | (0.39) | 0.17 | (0.31) |
| R_{free} | 0.20 | (0.31) | 0.19 | (0.31) | 0.28 | (0.43) | 0.20 | (0.37) |
| CC_{work} | 0.97 | (0.78) | 0.97 | (0.82) | 0.92 | (0.34) | 0.97 | (0.84) |
| CC_{free} | 0.96 | (0.77) | 0.96 | (0.83) | 0.90 | (0.43) | 0.97 | (0.70) |

| | | | | |
|---|---|---|---|---|
| Number of atoms without hydrogen | 4106 | 4104 | 22971 | 25064 |
| atoms for: | | | | |
| Macromolecules | 3627 | 3653 | 21616 | 21765 |
| Ligands | 79 | 146 | 198 | 214 |
| Solvent | 400 | 305 | 1157 | 3085 |
| Number of amino acids | 466 | 466 | 2796 | 2796 |
| RMS(bonds) | 0.009 | 0.008 | 0.008 | 0.006 |
| RMS (angles) | 1.03 | 1.01 | 1.02 | 0.93 |
| Ramachandran optimal (%) | 96.75 | 96.97 | 94.59 | 96.89 |
| Ramachandran allowed (%) | 3.25 | 3.03 | 4.87 | 3.11 |
| Ramachandran outliers (%) | 0.00 | 0.00 | 0.54 | 0.00 |
| Rotamer outliers (%) | 1.26 | 1.25 | 3.79 | 0.46 |
| Atomic overlapping index | 5.20 | 4.02 | 10.46 | 4.23 |
| Average B-factor for: | 24.02 | 23.22 | 44.93 | 19.03 |
| Macromolecules | 22.92 | 22.04 | 45.06 | 17.66 |
| Ligands | 29.32 | 32.70 | 47.49 | 18.50 |
| Solvent | 32.93 | 32.86 | 41.99 | 28.74 |

^{a}Statistical data for the highest resolution shell are given in parenthesis.

### Example 5

Study of the X-Ray Structure of PNP from H. pylori Complexes with Representative PNP Inhibitors

The resulting X-ray structure of PNP from H. pylori complexes with representative inhibitors of this enzyme: 6BnS-Pu, 2,6-diCl-Pu, 6BnO-2Cl-Pu and 6BnS-2Cl-Pu, were then analysed to characterise the interaction of the ligands with the active site of the enzyme. FIG. 3 shows a visualisation of the resulting crystal structure for the representative PNP inhibitor as described above, 6BnS-2Cl-Pu (Formula V), bound at the active site of the PNP from H. pylori. Hydrogen bonds (up to 3 Å) between ligands present in the PNP active site and water molecules and the enzyme are indicated by the dashed line. The structures of the other complexes were analysed similarly. Table 2 below summarises the data obtained for them, i.e. hydrogen bonds (up to 3 Å) and close contacts, i.e. not exceeding 3.5 Å.

TABLE 2
Hydrogen bonds (highlighted in bold) and close contact interactions shorter than 3.5 Å between
ligands and active site amino acids and water molecules observed in the studied complexes' structures.

| 6BnS—2Cl—Pu | | 6BnO—2Cl—Pu | | 6BnS—Pu | | 2.6-diCl—Pu | |
|---|---|---|---|---|---|---|---|
| N7 . . . 204(ASP)OD1 | 2.67 | N7 . . . 204(ASP)OD1 | 2.68 | N1 . . . 204(ASP)OD1 | 2.87 | N9 . . . 756(HOH)O | 2.78 |
| N9 . . . 302(TRS)N | 3.04 | N9 . . . 302(TRS)N | 2.94 | N7 . . . 215(HOH)O | 2.90 | N1 . . . 663(HOH)O | 2.86 |
| N1 . . . 192(HOH)O | 3.16 | C15 . . . 100(HOH)O | 2.99 | C11 . . . 942(HOH)O | 3.18 | C12 . . . 2(IMD)N1 | 3.15 |
| C8 . . . 203(SER)OG | 3.23 | C8 . . . 203(SER)OG | 3.22 | C11 . . . 159(PHE)O | 3.26 | C12 . . . 90(THR)O | 3.28 |
| CL1 . . . 158(PHE)O | 3.28 | CL1 . . . 158(PHE)O | 3.27 | N3 . . . 301(TRS)N | 3.27 | C12 . . . 204(ASP)OD2 | 3.36 |
| CL1 . . . 192(HOH)O | 3.34 | N7 . . . 203(SER)OG | 3.31 | C9 . . . 159(PHE)O | 3.28 | N3 . . . 2451(HOH)O | 3.51 |
| C9 . . . 159(PHE)O | 3.38 | C8 . . . 90(THR)OG1 | 3.38 | C9 . . . 215(HOH)O | 3.48 | C8 . . . 158(PHE)O | 3.53 |
| C9 . . . 192(HOH)O | 3.44 | CL1 . . . 100(HOH)O | 3.49 | | | C11 . . . 663(HOH)O | 3.53 |
| N7 . . . 203(SER)OG | 3.47 | | | | | | |
| C8 . . . 204(ASP)OD1 | 3.51 | | | | | | |

TRS—Tris buffer molecule, IMD—imidazole buffer molecule.
Bonds and contacts for the phosphate ion are not stated.

The results obtained confirm that the inhibitors tested bind to the active site of the PNP from H. pylori, meaning that their presence will result in inhibition of the reaction catalysed by this enzyme and thus may also inhibit the growth of H. pylori.

### Example 6

Analysis of the Inhibitory Properties of the PNP Enzyme from H. pylori by Exemplary PNP Inhibitors

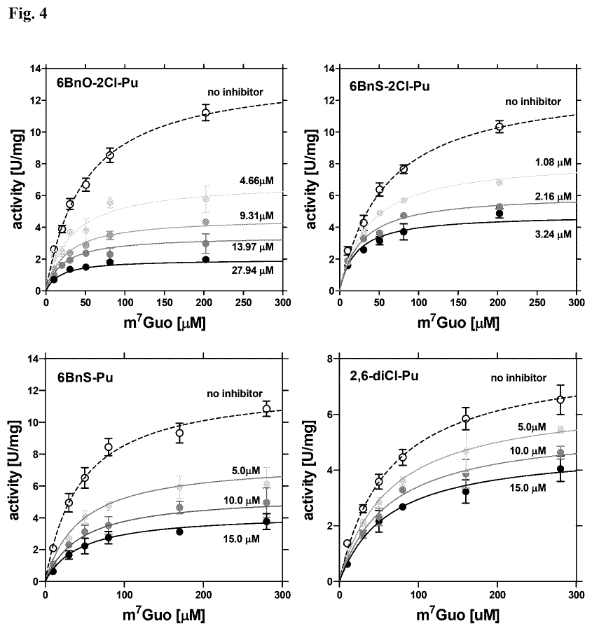

Study of the inhibition of the PNP enzyme from H. pylori by purines substituted at positions 2 and/or 6 for use according to the invention and by 2-chloro-6-benzylthiopurine-2′-deoxy-9-ribofuranoside was carried out using 7-methylguanosine as substrate at 25° C., in 50 mM Hepes/NaOH buffer pH 7.0, with a phosphate saturating concentration of 50 mM, measuring the progress of the reaction spectrophotometrically as described in Example 1. Data analysis was also performed as described in this Example. The range of substrate concentrations tested was 10-200 μM or 10-280 μM (shown in FIG. 4), and the range of inhibitor concentrations was chosen according to the observed degree of inhibition of the reaction, in order to obtain a significant degree of inhibition that allows determination of the inhibition mechanism and determination of inhibition constants (see FIG. 4).

For the inhibition studies of PNP from H. pylori, the compounds used were dissolved in methanol so that the concentration of the stock solution, which was then diluted with buffer, was 5 or 10 mM. The final concentration of methanol in the sample in which the enzyme activity was tested did not exceed 1%. By independent measurement, methanol at this concentration was shown to have a negligible effect on the rate of reaction catalysed by PNP from H. pylori. The inhibitor concentration was calculated from the weighed amount of compound and the volume of solvent in which it was dissolved and confirmed independently by absorbance measurement at pH 7.0 in water containing 10% methanol (v/v), using extinction coefficients ε₂₆₁ₙₘ=10,900 M⁻¹ cm⁻¹ for 6BnO-2Cl-Pu and ε₂₉₉ₙₘ=18,000 M⁻¹ cm⁻¹ for all 6-BnS-substituted compounds.

The enzyme activity data observed in the presence of the substrate but without the inhibitor and in the presence of different concentrations of the inhibitors tested are shown in FIG. 4. The data obtained were analysed similarly as described in Example 1. The inhibition model best describing the experimental data and the inhibition constants obtained by fitting the indicated model to the data are shown in Table 3 below.

TABLE 3
Inhibition properties of selected substituted purines with substitutions
at positions 2 and/or 6, and 2-chloro-6-benzylthiopurine-2′-deoxy-
9-ribofuranoside, against the PNP enzyme from H. pylori.

| | Kᵢₙ | K_{ic} | Kᵢᵤ | |
|---|---|---|---|---|
| Inhibitor | [μM] | [μM] | [μM] | alpha ^{a} |
| 2,6-dichloropurine ^{b} | 22.2 ± 1.4 | — | — | — |
| 2,6-dichloropurine | — | 20.0 ± 4.9 | 23.5 ± 3.5 | 1.17 ± 0.44 |
| 6-benzylthiopurine ^{b} | 7.9 ± 0.4 | — | — | — |
| 6-benzylthiopurine | — | 7.5 ± 1.3 | 8.1 ± 0.7 | 1.07 ± 0.27 |
| 2-chloro-6-benzyloxypurine ^{c} | — | 18.3 ± 7.3 | 4.6 ± 0.5 | 0.25 ± 0.12 |
| 2-chloro-6-benzyloxypurine | 6.5 ± 0.4 | — | — | — |
| 2-chloro-6-benzyloxypurine | — | — | 3.8 ± 0.3 | — |
| 2-chloro-6-benzylthiopurine ^{d} | — | — | 1.8 ± 0.2 | — |
| 2-chloro-6-benzylthiopurine | — | 13.2 ± 10.7 | 2.1 ± 0.3 | 0.16 ± 0.15 |
| 2-chloro-6-benzylthiopurine- | — | 6.2 ± 2.4 | 2.9 ± 0.5 | 0.47 ± 0.24 |
| 2′deoxy-9-ribofuranoside | | | | |
| 2-chloro-6-benzylthiopuryno- | — | 12.6 ± 5.7 | 2.0 ± 0.2 | 0.16 ± 0.08 |
| 2′-deoxy-9-rybofuranoside ^{c, e} | | | | |

^{a} alpha = Kᵢᵤ/K_{ic}
^{b} Kᵢₙ -The non-competitive model best describes the experimental data obtained
^{c} The mixed type inhibition model best describes the experimental data obtained
^{d} The uncompetitive and the mixed-type inhibition models have similar probability. Due to the poor solubility of the compound, the data are limited to low concentrations of the inhibitor, the errors of K_{ic} and the alpha parameter are high, so it is not possible to clearly state which inhibition model is better
^{e} Inhibition for guanosine as a substrate

The data obtained confirm that the compounds tested inhibit the PNP enzyme from H. pylori, wherein 6BnS-2Cl-Pu is the strongest inhibitor. The data obtained are best described by the uncompetitive model, and the inhibition constant characterising the interaction of this compound with the enzyme is 1.8±0.2 μM.

### Example 7

Study of Inhibition of H. pylori Cell Growth in Bacterial Culture by Inhibitors from Example 6

Having confirmed the inhibitory properties against PNP from H. pylori of the compounds for use according to the invention, the present inventors carried out tests to confirm whether the compounds inhibit the growth of H. pylori in culture. The representative PNP inhibitors from H. pylori included in this study were: 2,6-diCl-Pu, 6BnO-2Cl-Pu, 6BnS-2Cl-Pu, 6BnS-Pu, 6BnS-2Cl-9dr.

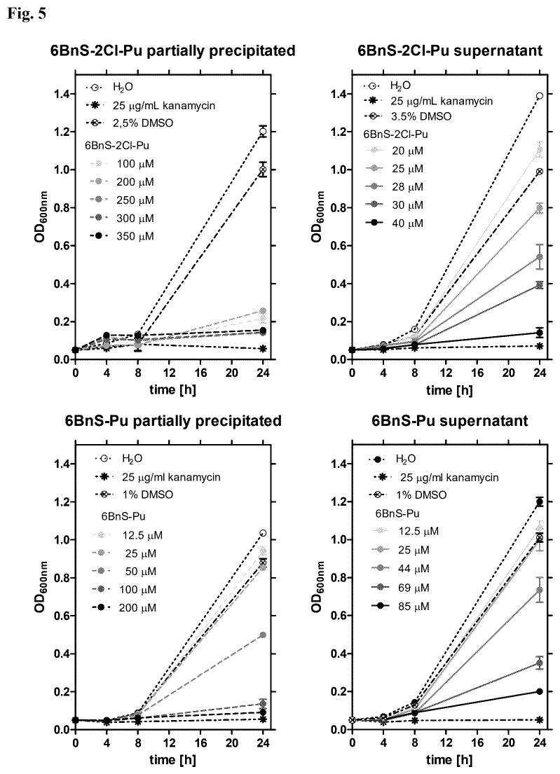

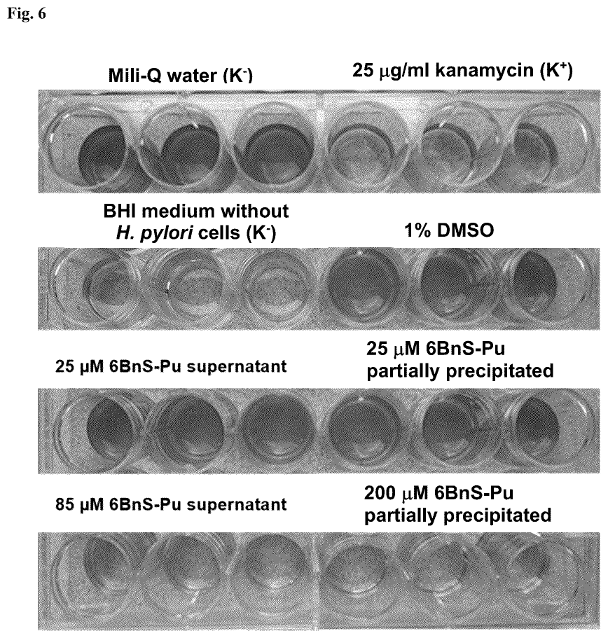

The experiments were carried out using the growth curve method as described in Example 3, and the results obtained for two of the compounds tested are shown in FIG. 5. As the water solubility of the compounds tested, with the exception of 2,6-diCl-Pu, is relatively poor, two experiments were carried out: the first with the maximum concentration possible under the experimental conditions in aqueous solution with a small admixture of DMSO, and the second with higher concentrations that led to partial precipitation of the compound. The aim of the second experiment was to demonstrate that the compound could be administered in a partially undissolved form without compromising the inhibitory effect on H. pylori proliferation in cultures.

On the basis of the experimental data obtained for all the compounds mentioned in the first paragraph, the values of the minimum inhibitory concentration, MIC, were determined and are shown in Table 4 below. This table also includes, for comparison, the literature MIC data for the other antibiotics, metronidazole and kanamycin, and the data for these antibiotics obtained by the inventors on the H. pylori strain 26695. It can be seen that compound 6BnS-2Cl-Pu at a concentration of 40 μM shows a 92% inhibition of the growth of the H. pylori strain 26695, thus comparable to the inhibition of the same strain by metronidazole at a concentration of 12 μM and kanamycin at a concentration of 52 μM.

TABLE 4
Minimum inhibitory concentration (MIC) values and inhibition achieved
at maximum solubility in 3.5% DMSO of the compounds tested in this
experiment against cultures of the H. pylori strain 26695.
Results are presented as the average of three repeats.

| | | | | | | % inhibition of |
|---|---|---|---|---|---|---|
| | | | | | | H. pylori cell |
| | | | | | | cultures at |
| | | | | Maximum | | maximum |
| | | MIC ^{a} | | solubility | | inhibitor |
| Inhibitor | MW | [μg ml⁻¹] | [μM] | [μg ml⁻¹] | [μM] | solubility |
| DMSO | 78.13 | — | — | 55.0 ^{b} | — | 60 ^{e} |
| | | | | 38.5 ^{b} | — | 26 ^{e} |
| | | | | 27.5 ^{b} | — | 0 ^{e} |
| 2,6-diCl—Pu | 189.00 | 473 | 2500 | 236 | 1250 | 93 |
| 6BnO—2Cl—Pu | 260.68 | — | — | 25 ^{c} | 95 ^{c} | 35 |
| | | | | 91 ^{d} | 350 ^{d} | 75 |
| 6BnS—2Cl—Pu | 276.74 | 69 ^{d} | 250 ^{d} | 11.1 | 40 | 92 |
| 6BnS—Pu | 242.3 | 48 ^{d} | 200 ^{d} | 21 | 85 | 83 |
| 6BnS—2Cl—Pu-9dr | 393.87 | 79 ^{d} | 200 ^{d} | 4.3 ^{c} | 10.8 ^{c} | 5 |
| Metronidazole | 171.15 | 8 ^{f} | 47 | 2 ^{g} | 12 ^{g} | 89-92 |
| Kanamycin | 484.5 | 0.5-64 ^{f} | 1.1-132 | 25 ^{g} | 52 ^{g} | 92-96 |
| BHI medium | — | — | — | — | — | 95 ^{h} |
| without H. pylori | | | | | | |

^{a} The MIC is the minimum concentration used in a series of two-fold dilutions that gave full growth inhibition of H. pylori cultures, i.e. comparable to that observed for the positive control with 25 μg/ml kanamycin, i.e. 92-96%.
^{b} This corresponds to 5%, 3.5% and 2.5% (v/v), respectively, and in this case this is not the maximum solubility; density of DMSO: 1.1 g/cm³
^{c} This was the maximum achievable concentration of the inhibitor under the conditions (saturation was observed at this concentration)
^{d} The inhibitor has partially precipitated, so the actual concentration of the dissolved compound is lower
^{e} In each experiment, the effect of DMSO alone at the maximum concentration used in that experiment was independently tested. This was necessary because the inhibition observed for a given % DMSO varies slightly between experiments
^{f} MTZ according to EUCAST, 2021, kanamycin according to Irie et al., 1997
^{g} In this case, this concentration is not the concentration at maximum solubility
^{h} In this case, there are no H. pylori bacteria, and therefore such a sample is treated as a negative control.

The experiments allowed to conclude that all the inhibitors tested over a wide range of concentrations inhibited the growth of H. pylori in culture. The best inhibitors proved to be 6BnS-2Cl-Pu and its precursor, 6BnS-2Cl-Pu-9dr. The most potent inhibitor, 6BnS-2Cl-Pu, showed almost complete inhibition of bacterial growth (92%, thus comparable to that induced by kanamycin and metronidazole, FIG. 5, Table 4) at a concentration of 40 μM, the maximum obtainable under the conditions. 6BnS-2Cl-Pu-9dr at a concentration of 200 μM, although partially precipitated, allowed the observation of complete growth inhibition of H. pylori strain 26695 (Table 4). Complete inhibition of the growth of H. pylori strain 26695 can also be achieved with the 6BnS-Pu inhibitor solution at a concentration of 200 μM, although it undergoes partial precipitation at this concentration (FIGS. 5 and 6).

### Example 8

Study of the Effect of the Use of a PNP Inhibitor in Combination with an Antibiotic

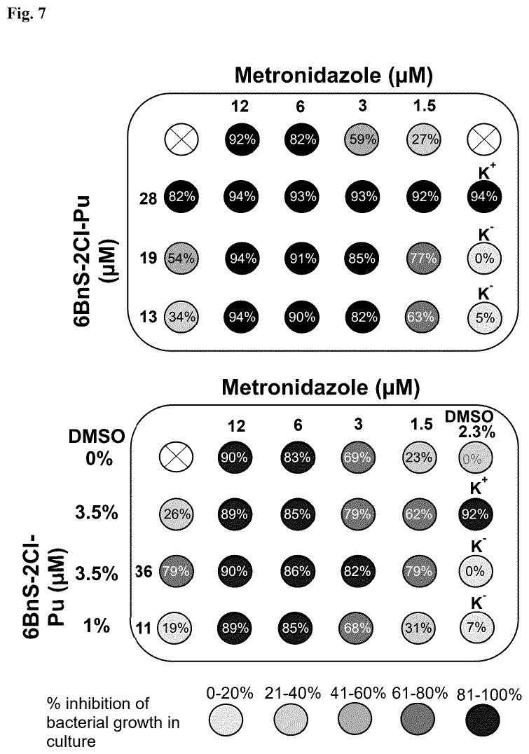

In this study, a representative inhibitor of PNP enzyme activity from H. pylori, 6BnS-2Cl-Pu, was used in combination with a representative antibiotic used for the treatment of H. pylori infection, metronidazole (MTZ), to test whether this combination provides an additive or synergistic effect. The study was conducted using the so-called checkerboard test (Krzyżek et al., 2019). Both compounds were tested at concentrations below the MIC. The concentration gradient used for 6BnS-2Cl-Pu was 11-36 μM (3-10 μg/ml), chosen on the basis of the results from Example 7. The concentration range of MTZ was chosen on the basis of the MIC value reported in EUCAST (2021), i.e. 47 μM (8 μg/ml). However, in the case of H. pylori strain 26695, 89-92% inhibition was observed already at 12 μM (2 μg/ml) (Table 4), so concentrations of no more than 12 μM were used. Solutions of the tested compounds were prepared in test tubes in such a way as to obtain twice the tested concentrations when testing the activity of 6BnS-2Cl-Pu alone or metronidazole alone, and four times the test concentrations when testing a combination of both compounds. The experiment was conducted similarly to that described in Example 3 for the determination of MIC values, but 125 μl of each compound at four-fold test concentrations was added to the wells in which the activity of both compounds present simultaneously was determined and supplemented with 250 μl of inoculated BHI medium at double strength (OD₆₀₀ approximately 0.05, corresponding to 10⁴-10⁵ CFU/ml). Since the 6BnS-2Cl-Pu solutions were prepared with the admixture of DMSO, the effect of this compound, at an analogous concentration, was also checked. The data obtained in the experiments are summarised in FIG. 7.

The interaction between the compounds tested was determined by calculating the fractional inhibitory concentration index (FICI) according to Pillai et al. (2005). The data obtained, presented in the upper panel of FIG. 7, suggest the occurrence of an additive effect of the antibiotic used in combination with the PNP inhibitor (metronidazole in combination with 6BnS-2Cl-Pu), since the FICI value calculated on the basis of these data is 0.83. This value, as it is contained in the range between 0.5 and 1.0, indicates the additive effect.

This confirms the possibility of using an inhibitor of the H. pylori cell salvage pathway, in combination with one of the antibiotics currently used in H. pylori eradication, to treat H. pylori infection. By using such a combination, the efficacy of the treatment of H. pylori infection can be increased, the amount of antibiotic used can be reduced and the risk of the emergence of antibiotic-resistant strains can be reduced.

### Example 9

Study of the Effect of PNP Inhibition on the Growth of Drug-Resistant H. pylori

In this study, the effect of selected purine nucleoside phosphorylase (PNP) inhibitors of the cellular purine nucleotide salvage pathway (mefloquine and quinine) on the growth of antibiotic-resistant H. pylori strains, i.e. those resistant to clarithromycin (CLR) and metronidazole (MTZ), or both antibiotics simultaneously, was analysed by determining the minimum inhibitory concentration (MIC) values.

TABLE 5
Data describing the antibiotic-resistant H. pylori strains tested

| H. pylori | Patient's | Patient's | | H. pylori |
|---|---|---|---|---|
| strain | sex | age | Diagnosis | resistance |
| M26 | K | 63 | Reflux disease | MTZ + CLR |
| M91 | M | 82 | Gastroenteritis and duodenitis | MTZ + CLR |
| M92 | K | 63 | Duodenal ulcer | CLR |
| M93 | K | 62 | Gastroenteritis and duodenitis | MTZ |

The antibacterial activity of two compounds, mefloquine (in the form of hydrochloride) and quinine, described in the literature as potent in vitro inhibitors of the PNP enzyme from Plasmodium falciparum (Dziekan et al., 2019), was tested. Using a two-fold serial dilution method in liquid medium (as described in Example 3), the MIC values for mefloquine and quinine were determined against clinical H. pylori strains resistant to MTZ, CLR and MTZ+CLR (M26, M91, M92 and M93), as well as the reference H. pylori strain 26695. Four clinical H. pylori strains isolated from adults, from primary infections (the subjects had never previously been treated against H. pylori infection) were tested. These strains come from the collection of the Department of Microbiology of the Medical University of Wroclaw and were obtained due to collaboration with Prof. Grażyna Gościniak, MD, PhD. The tested compounds belong to organic substances and are soluble in DMSO. The maximum tested concentration of quinine in H. pylori culture was 0.56 mM, and that of mefloquine was 2.25 mM. The final DMSO concentration did not exceed 2.5%. On each occasion, the effect of DMSO alone on the growth of the H. pylori strains tested was also checked. In the case of mefloquine, more dilutions of the compound were made than in the case of quinine, up to the lowest tested concentration of 9 μM, because mefloquine was found to effectively inhibit the growth of all tested H. pylori strains even at low concentrations, where the concentration of DMSO is negligible and therefore neutral to the bacterial cells. Kanamycin was used as a positive control and Mili-Q deionised water as a negative control. All experiments were carried out in triplicate.

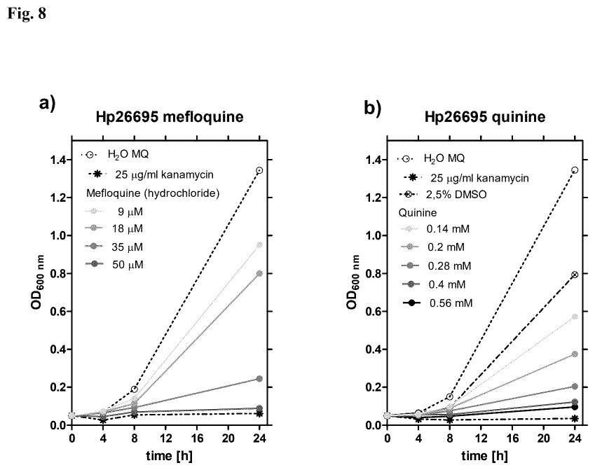

The results of the preliminary experiments showed that mefloquine effectively inhibited the growth of the reference H. pylori strain 26695 (MIC=50 μM or 21 μg/ml), while quinine inhibited the growth of this bacterium at much higher concentrations (MIC ≥0.4 mM; MIC ≥130 μg/ml) (FIG. 8a, b). The percentage of growth inhibition of H. pylori at this quinine concentration is 91%. However, DMSO at a concentration of 2.5% significantly inhibits the growth of the H. pylori strain 26695 (41%), so the effect of quinine on the growth of H. pylori at the highest concentration tested of 0.56 mM cannot be determined. The preliminary results obtained are very promising and have inspired the investigation of whether mefloquine and quinine will also inhibit the growth of resistant H. pylori strains so difficult to eradicate.

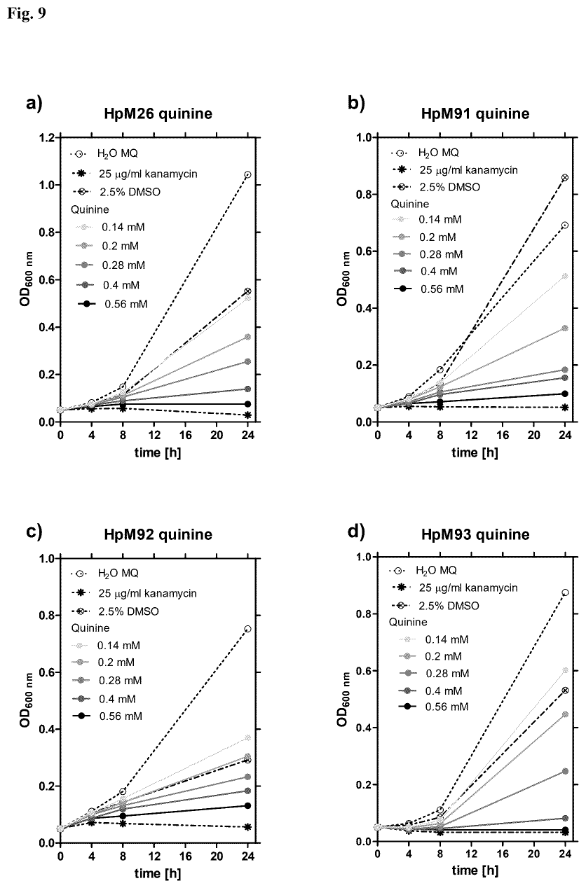

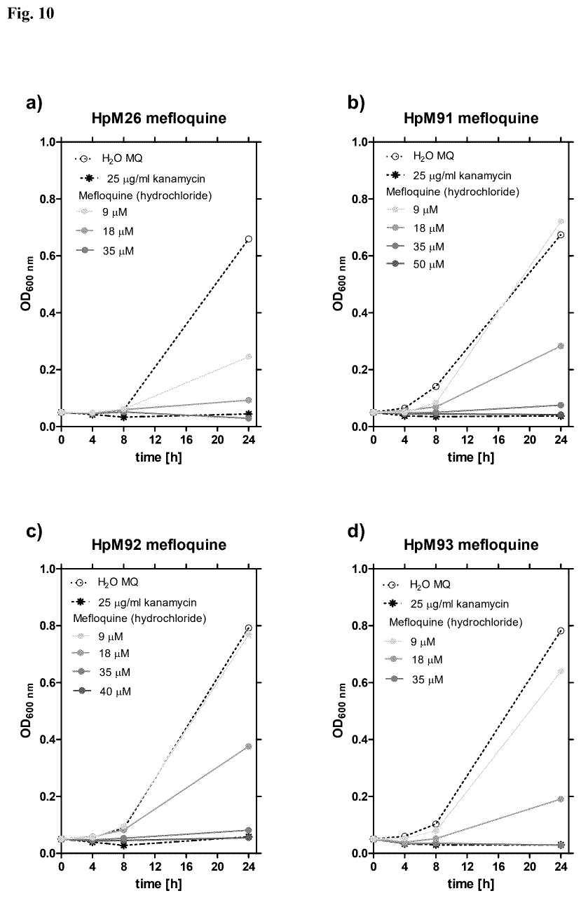

The growth curves of H. pylori strains resistant to clarithromycin (c), metronidazole (d) and to both antibiotics simultaneously (a, b) in the presence of different concentrations of quinine and mefloquine are shown in FIGS. 9 and 10. The MIC values determined from these results are summarised in Table 5. In the case of quinine, the MIC value against strains M26, M92 and M93 is ≥0.4 mM (≥130 μg/ml), and the percentage of growth inhibition of H. pylori at this concentration of the compound was respectively: 87%, 76% and 91% (FIG. 9a, c, d). The determined MIC value is the same as for the reference H. pylori strain 26695. When these bacterial strains were tested, DMSO at a concentration of 2.5% also proved to significantly inhibit their growth (47%, 61% and 39%, respectively), so the effect of quinine on the growth of H. pylori at the highest concentration tested, 0.56 mM, could not be determined. In contrast, when strain M91 was tested, 2.5% DMSO was found to be neutral to its growth. The percentage of growth inhibition of H. pylori at the highest tested concentration of the compound 0.56 mM is 86% and at a concentration of 0.4 mM 78%. Therefore, the MIC value for this strain was set at ≥0.56 mM (≥182 μg/ml) (FIG. 9b). Mefloquine was tested at the same concentrations as for H. pylori strain 26695. The MIC value was determined for strain M92 (40 μM=17 μg/ml) and for strain M91 (50 μM=21 μg/ml), for which the value was the same as for the reference H. pylori strain 26695 (FIG. 10c, b). The MIC against strains M26 and M93 was shown to be 35 μM (14.5 μg/ml), the lowest value among those determined (FIG. 10a, d). Mefloquine is very effective in inhibiting the growth of clarithromycin- and metronidazole-resistant H. pylori strains at much lower concentrations compared to quinine. For some strains (M26 and M93), the MIC value for mefloquine is even lower than that determined for the reference H. pylori strain 26695. In order to analyse the results obtained, a comparison was made between the determined MIC values for mefloquine and quinine and their maximum doses of these drugs used in malaria treatment (Table 6), which were defined in the latest WHO guidelines.

TABLE 6
Comparison of the MIC values of quinine and mefloquine obtained for the tested resistant
H. pylori strains with the maximum doses of these compounds used in malaria treatment.

| | Maximal | MIC value for tested resistant strains of H. pylori | | | |
|---|---|---|---|---|---|
| Inhibitor | dose | M26 | M91 | M92 | M93 |
| Quinine | 10 mg/kg | ≥0.4 mM | ≥0.56 mM | ≥0.4 mM | ≥0.4 mM |
| | of the body | (≥130 μg/ml) | (≥182 μg/ml) | (≥130 μg/ml) | (≥130 μg/ml) |
| | weight | | | | |
| | every 8 | | | | |
| | hours | | | | |
| Mefloquine | 25 mg/kg | 35 μM | 50 μM | 40 μM | 35 μM |
| (hydro- | of the body | (14.5 μg/ml) | (21 μg/ml) | (17 μg/ml) | (14.5 μg/ml) |
| chloride) | weight per | | | | |
| | whole | | | | |
| | therapy | | | | |

Based on the studies conducted, mefloquine inhibits the growth of H. pylori even at very low concentrations. Quinine also inhibits the growth of H. pylori, and therefore both compounds could be used in the treatment of H. pylori infection and diseases associated with H. pylori infection, particularly those associated with infection with H. pylori strains resistant to currently used antibiotics such as clarithromycin and metronidazole.

### Example 10

Study of AdSS Enzyme from H. pylori Activity in the Presence of an Inhibitor-Pyridoxal 5′-Phosphate (PLP) and its Metabolic Precursor, Pyridoxal

This study assessed the interaction of pyridoxal 5′-phosphate (PLP) and its metabolic precursor, pyridoxal (PI-h), with AdSS from H. pylori strain 26695.

### Enzyme Activity Assay

Enzymatic activity measurements were carried out in the similar manner as described in Bubić et al. (2018), at 25° C., in cuvettes with an optical path length of 1 cm containing 1 ml of reaction mixture consisting of 20 mM Hepes/NaOH buffer pH 7.7, 1 mM MgSO4 and the substrates, GTP, IMP and Asp. Saturating concentrations of substrates, namely GTP: 0.06 mM, IMP: 0.15 mM and Asp: 5 mM, were used to measure specific activity. The extinction coefficient at 280 nm of 1.17×10⁴ M⁻¹ cm⁻¹ (adenylosuccinate formation) was used to calculate the concentration of the product formed (Rudolph and Fromm 1969). One unit (U) of AdSS specific activity is defined as μmol of adenylosuccinate produced per minute at 25° C. Specific activity is expressed as units of enzymatic activity per mg of protein (U/mg).

### 10a) Dependence of AdSS Inactivation by PLP on Inhibitor Concentration

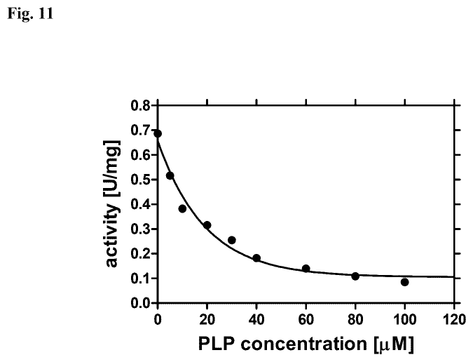

Inhibition of AdSS by PLP was examined first at saturation with all enzyme substrates and with increasing concentrations of the PLP inhibitor. The data obtained are shown in FIG. 11. These results confirm that at concentrations of the inhibitor, PLP, above about 100 μM, enzyme activity decreased by 86%, with no further decrease observed, while the PLP concentration leading to half of this inhibition was 13.3 μM.

10b) Inactivation of AdSS from H. pylori by PLP as a Function of Time

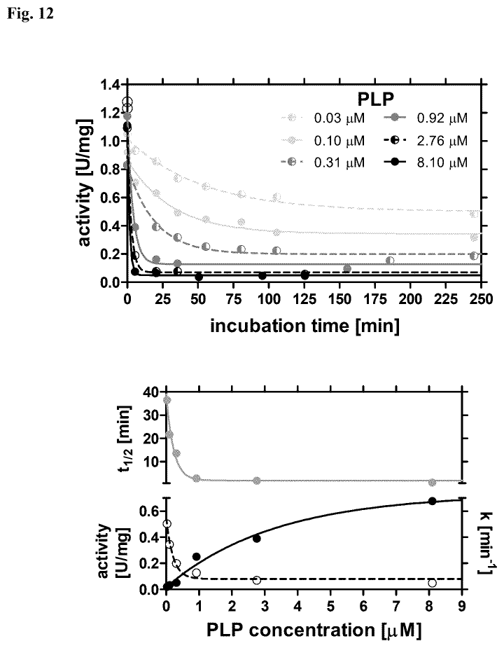

To determine the extent to which PLP-induced inactivation develops over time, the enzyme at low concentration (0.022 μM) was incubated at 25° C. in Hepes/NaOH buffer pH 7.0, in the presence of 1.11 mM MgSO₄, 5.55 mM Asp, 167 μM IMP and PLP at various concentrations (ranging between 0.03 μM and 8.1 μM). The mixture contained all components necessary for the reaction with the exception of GTP, which, according to Dong and Fromm (1990), competes with PLP. Aliquots of 900 μl were taken from the incubated mixture at specified time intervals, placed in a cuvette and 100 μl of 0.6 mM GTP solution was added to initiate the reaction. The enzyme activity was determined and analysed in the known manner as described above. The results obtained are shown in FIG. 12.

Under the conditions tested, when the inhibitor was able to interact with the enzyme in the absence of substrate, GTP, even at the lowest concentrations tested, namely 0.03 μM, PLP led to more than 50% inactivation of AdSS from H. pylori. The activity observed in the absence of PLP was 1.23 U/mg, falling to 0.48 U/mg after about 4 hours of incubation, as shown in the upper panel of FIG. 12. As the PLP concentration increased, the inactivation process proceeding faster and faster. PLP present at a concentration of 8.1 μM, causes a decrease in activity to 0.04 U/mg, thus by about 97% after only about 10 minutes.

Exponential decays were fitted to the data obtained for each concentration and the reaction rate constant of the inactivation process, k, and the residual enzymatic activity observed when the system reached equilibrium, A_{∞}, were obtained as fitting parameters. From these parameters, the half-life of the inactivation process activity, t_{1/2}, was calculated. The dependence of these three parameters on PLP concentration is shown in the lower panel of FIG. 12.

The data obtained show that PLP is a potent inhibitor of AdSS from H. pylori. Under experimental conditions, already at a concentration of 1 μM, it causes almost complete, specifically 90%, inactivation of the enzyme.

10c) Dependence of AdSS Activity on the Concentration of the PLP Precursor i.e. Pyridoxal

The inhibition of AdSS from H. pylori by pyridoxal (PI-h) was studied at saturation with all enzyme substrates and with increasing concentrations of the inhibitor, similar to that for PLP in Example 10a). In contrast to PLP, its metabolic precursor PI-h even at the maximum concentration used, i.e. 400 μM, has no effect on AdSS activity. This result shows that pyridoxal requires phosphorylation to bind strongly to AdSS from H. pylori.

### Example 11

Growth Inhibition of H. pylori Strains by Representative AdSS Inhibitors: PLP and its Metabolic Precursors

In this study, the effects of pyridoxal 5′-phosphate (PLP) and its metabolic precursors, at concentrations up to 5 mM, on the replication of three representative H. pylori strains, 26695, N6 and P12, were studied to determine the minimum inhibitory concentration (MIC) for the aforementioned H. pylori strains.

Inhibition by PLP and its metabolic precursors of the growth of H. pylori strains: 26695, N6 and P12 in culture was investigated using the growth curve method as described in Example 3. From these experiments, the minimum inhibitory concentration was determined as the lowest concentration of a compound that inhibits the visible growth of the bacteria, compared to growth in control samples without the tested compounds. All experiments were carried out in triplicate.

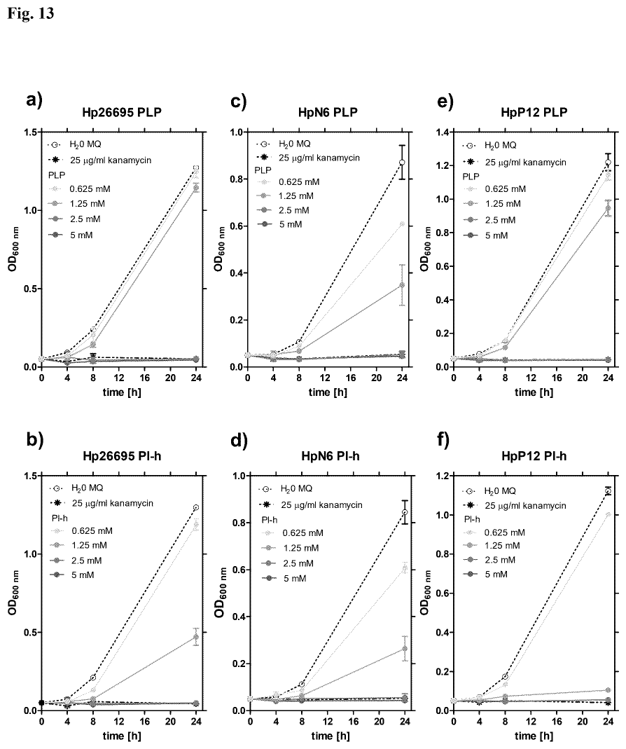

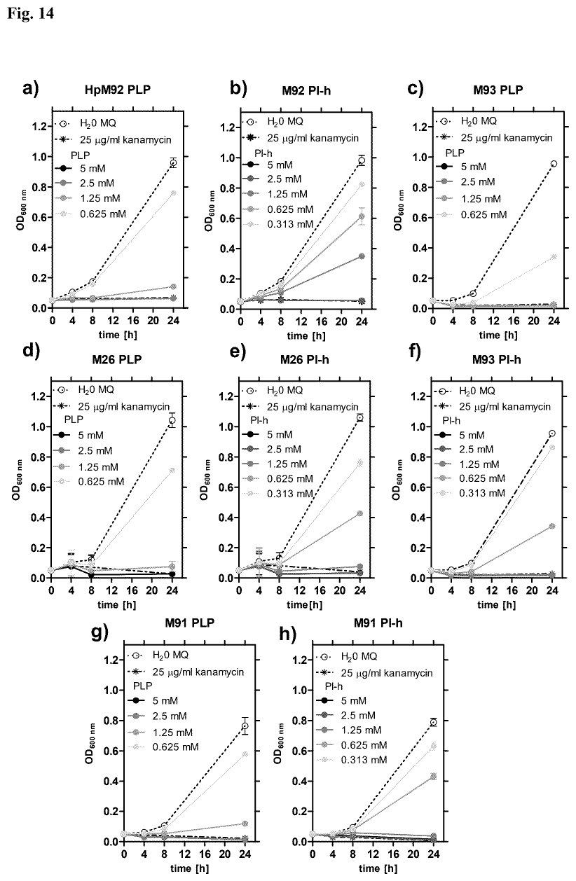

The tests included pyridoxal 5′-phosphate (PLP) and its precursors: pyridoxal hydrochloride (PI-h), pyridoxine, pyridoxine in the form of hydrochloride and pyridoxamine dihydrochloride. Apart from the first two compounds, no inhibition of bacterial growth was observed (data not shown). In contrast, PLP and PI-h in a similar manner slowed the growth of all H. pylori strains tested (FIG. 13), with determined MIC values of 618 μg/ml (2.5 mM) for PLP and 509 μg/ml (2.5 mM) for PI-h. The results thus confirm the utility of PLP and its metabolic precursor in inhibiting the growth of H. pylori.

These results, together with the results of Example 10a) showing the lack of effect of PI-h on the AdSS enzyme itself, demonstrate that pyridoxal is phosphorylated to PLP in H. pylori cells, and only in this form does it inhibit bacterial proliferation.

### Example 12

Growth Inhibition of Drug-Resistant H. pylori Strains by a Representative AdSS Inhibitor, PLP, and its Metabolic Precursor

In this study, the effects of pyridoxal 5′-phosphate (PLP) and its precursor, i.e. pyridoxal (in the form of hydrochloride, PI-h), at concentrations up to 5 mM, on the replication of four representative antibiotic-resistant strains of H. pylori, resistant to clarithromycin, to metronidazole, or to both antibiotics simultaneously, were determined. These strains are described in more detail in Example 9.

The inhibition by PLP and PI-h of the growth of H. pylori strains in culture was tested using the growth curve method, as described in Examples 3, 9 and 11. From these experiments, the minimum inhibitory concentration, MIC, was determined for both derivatives tested, as the lowest concentration of the compound that inhibits visible bacterial growth, compared to growth in control samples without the compounds tested. All experiments were carried out in triplicate. The results for all tested pairs are shown in FIG. 14, and the MIC values determined from them are summarised in Table 7.

TABLE 7
Comparison of the minimum inhibitory concentration (MIC) values
of PLP and PI-h for wild-type and metronidazole (MTZ) and/or
clarithromycin (CLR) resistant H. pylori strains.

| H. pylori | | MIC for PLP | MIC for PLP | MIC for PI-h | MIC for PI-h |
|---|---|---|---|---|---|
| strain | Resistance | [μM] | [μg ml⁻¹] | [μM] | [μg ml⁻¹] |
| 26695 | — | 2.5 | 618 | 2.5 | 509 |
| N6 | — | 2.5 | 618 | 2.5 | 509 |
| P12 | — | 2.5 | 618 | 2.5 | 509 |
| M92 | CLR | ≥1.25; ≤2.5 | ≥309; ≤618 | 2.5 | 509 |
| M93 | MTZ | 1.25 | 309 | 1.25 | 255 |
| M26 | MTZ + CLR | 1.25 | 309 | 1.25 | 255 |
| M91 | MTZ + CLR | ≥1.25; ≤2.5 | ≥309; ≤618 | 1.25 | 255 |

The MIC values obtained against resistant strains were compared with the analogous values determined in Example 11 for the three wild-type H. pylori strains tested. As it turns out, the tested antibiotic-resistant H. pylori strains generally show greater sensitivity to PLP and its precursor, PI-h, than the wild-type H. pylori strains, since only for the PI-h pair and strain M92 the determined MIC value is 509 μg/ml (2.5 mM), which is the same as the MIC values for this compound for the three tested wild-type strains. Meanwhile, for two of the resistant strains tested, M26 and M93, the MIC values for both compounds, and for strain M26 for PI-h, are twice as low as for the wild-type strains, at 309 μg/ml (1.25 mM) and 255 μg/ml (1.25 mM) for PLP and PI-h, respectively.

### Example 13

Obtaining and Analysing the X-Ray Structure of a Ternary Complex of AdSS from H. pylori with IMP and Pyridoxal 5′-Phosphate

The crystal structure of the ternary complex of the enzyme under study, AdSS from H. pylori, with pyridoxal 5′-phosphate (PLP) and one of the substrates, IMP, was determined by X-ray diffraction, and analysed.

Crystals of the PNP complex with AdSS and IMP were obtained at 12° C. using both the hanging drop method under conditions of 0.1 M Tris/HCl pH 8.5, 0.5 M Am₂SO₄, 29% PEG 3350 and the capillary method developed by the present inventors under conditions of 85 mM Tris/HCl, 21% PEG 4k, 170 mM Li₂SO₄, 15% glycerol. As the structures obtained from the two conditions do not differ, one with the best performance (resolution 1.85 Å, R_{work} 0.166, R_{free} 0.201) is described in the following. AdSS enzyme from H. pylori with a His-Tag at the C-terminus was obtained as described (Bubić et al., 2023). The protein in 20 mM Hepes/NaOH buffer pH 7.0, 150 mM NaCl, 2 mM TCEP was complexed with PLP and IMP, with a final enzyme concentration of 15 mg/ml and PLP and IMP concentrations of 7.5-fold and 6.25-fold higher, respectively. Capillaries for X-ray measurements with diameters of 0.3 mm or 1 mm (Glaskapillaren Mark-Rohrchen fur rontgenographischen Aufnamen, GLAS, W. Muller) were filled with the protein-ligand complex to a certain height, followed by the addition of 2-fold the volume of crystallisation liquid. The capillaries were sealed with wax on both sides.

Diffraction measurements on selected enzyme crystals were performed on a SuperNova X-ray diffractometer with a copper lamp (2=1.541838 Å) and an Atlas CCD camera (distributor Oxford Diffraction, Rigaku).

Data were integrated using CrysAllis software (Oxford Diffraction/Rigaku, software provided by the diffractometer manufacturer). All structures were solved by molecular replacement using Phaser (McCoy, 2007), using the structure of H. pylori AdSS in complex with IMP (PDB code 7PVO, Bubić et al., 2023) as a model. Models were refined using RefMac5 (Murshudov et al., 2011) and Coot (Emsley et al., 2010). Statistical information for the collected data and parameters of the obtained structure, after its refinement, are summarised in Table 8. The asymmetric unit comprises one monomer of the enzyme.

TABLE 8
Collected diffraction data and their statistical analysis
and parameters of the obtained structure.

| | Complex of AdSS from H. pylori with | |
|---|---|---|
| Structure | PLP and IMP | |
| Resolution range (Å) | 29.80-1.85 | (1.89-1.85) ^{a} |
| Space group | I 1 2 1 | |
| Unit cell | | |
| a, b, c (Å) | 68.897 61.124 119.254 | |
| α, β, γ (°) | 90.000 98.905 90.000 | |
| Total number of reflections | 984730 | (32859) |
| Number of unique reflections | 41958 | (2580) |
| Multiplicity | 45069 | (45119) |
| Completness (%) | 100.0 | (100.0) |
| Mean I/σ(I) | 48.0 | (44993) |
| Wilson B factor | 12.36 | |
| R_{merge} | 0.049 | (0.251) |
| Rₘₑₐₛ | 0.05 | (0.261) |
| Rₚᵢₘ | 0.01 | (0.073) |
| CC_{1/2} | 1.000 | (0.991) |

| | |
|---|---|
| Reflections used for refinement | 41870 |
| Reflections used for calculation of R_{free} | 4135 |
| R_{work} | 0.166 |
| R_{free} | 0.201 |
| Number of atoms without hydrogen atoms | |
| for: | |
| Macromolecules | 3199 |
| Ligands | 49 |
| Solvent | 267 |
| Number of amino acids | 396 |
| RMS(bonds) | 0.0103 |
| RMS (angles) | 1.759 |
| Ramachandran optimal (%) | 386 |
| Ramachandran allowed (%) | 16 |
| Ramachandran outliers (%) | 1 |
| Rotamer outliers (%) | 13 |
| Atomic overlapping index | 4.6 |
| Average B-factor for dla: | |
| Macromolecules | 19.29 |
| Ligands | 29.56 |
| Solvent | 26.7 |

^{a} Statistical data for the highest resolution shell are given in parenthesis

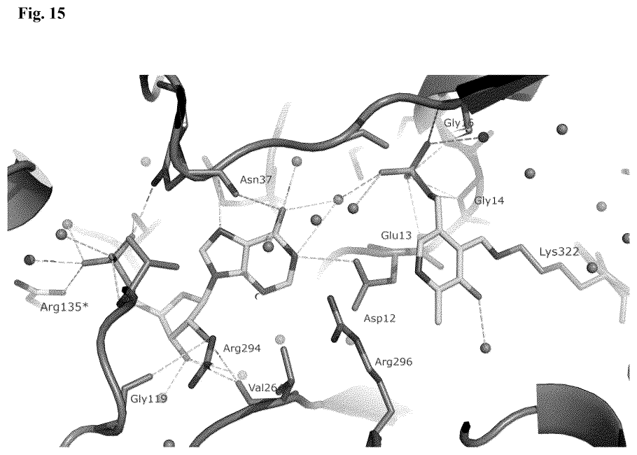

The resulting X-ray structure of the ternary complex of AdSS from H. pylori with PLP and IMP was then analysed to characterise the PLP binding site and details of ligand interactions with the enzyme active site. FIG. 15 shows a visualisation of the resulting crystal structure of AdSS from H. pylori, as described above, with PLP and IMP bound to the enzyme active site. Hydrogen bonds (up to 3 Å) between ligands present in the active site of AdSS and water molecules and the enzyme are indicated by a dashed line.

It has turned out that the IMP molecule in the ternary complex of AdSS with PLP and IMP, is bound in the active site of the enzyme in an identical position to that in the binary complex of AdSS with IMP alone, while PLP occupies a fragment of the active site dedicated to the second of the enzyme's substrates, GTP, and forms a covalent bond with the enzyme via the Lys322 side chain.

In conclusion, it is important to note that the studies carried out have shown that inhibitors of the H. pylori cell purine nucleotide salvage pathway enzyme, especially inhibitors of the PNP enzyme and/or the AdSS enzyme, as well as metabolic precursors of some inhibitors that do not inhibit these enzymes on their own or inhibit them weakly, only interacting strongly with the enzyme after conversion to the phosphorylated active form, effectively inhibit the growth of H. pylori, either alone or in combination, and can therefore be used effectively in the treatment of H. pylori infection, as well as in the treatment and/or prevention of diseases associated with H. pylori infection. In addition, these inhibitors can be used in combination with other drugs to treat H. pylori infection, thus providing novel combination therapies for use in the treatment of H. pylori infection and/or in the treatment or prevention of diseases associated with H. pylori infection.

### REFERENCES

Bubić A, Mrnjavac N, Stuparević I, Łyczek M, Wielgus-Kutrowska B, Bzowska A, Luić M, Leščić Ašler I (2018) In the quest for new targets for pathogen eradication: the adenylosuccinate synthetase from the bacterium Helicobacter pylori J. Enzyme Inhib. Med. Chem. 33:1405-1414, doi: 10.1080/14756366.2018.1506773
Bubić A, Narczyk M, Petek A, Wojtys M I, Maksymiuk W, Wiekgus-Kutrowska B, Winiewska-Szajewska, M, Pavkov-Keller T, Bertoša B, Štefanić Z, Luić M, Bzowska A, Leščić Ašler I (2023) The pursuit of new alternative ways to eradicate Helicobacter pylori continues: Detailed characterization of interactions in the adenylosuccinate synthetase active site. Int. J. Biol. Macromol. 226:37-50. doi: 10.1016/j.ijbiomac.2022.12.001
Bzowska A, Kulikowska E, Shugar D (1990) Properties of purine nucleoside phosphorylase (PNP) of mammalian and bacterial origin, Z. Naturforsch. 45c: 59-70. doi: 10.1515/znc-1990-1-211
Bzowska A, Kulikowska E, Shugar D (1992) Formycins A and B and some analogues: selective inhibitors of bacterial (Escherichia coli) purine nucleoside phosphorylase. Biochim Biophys Acta (BBA)/Protein Struct Mol 1120:239-247. doi: 10.1016/0167-4838 (92) 90243-7
Bzowska A, Kulikowska E, Shugar D (2000) Purine nucleoside phosphorylase: properties, functions and clinical aspects. Pharm. Ther. 88:349-425. doi: 10.1016/s0163-7258 (00) 00097-8
Bzowska A, Magnowska L, Kazimierczuk Z (1999) Synthesis of 6-Aryloxy- and 6-Arylalkoxy-2-Chloropurines and Their Interactions with Purine Nucleoside Phosphorylase from Escherichia Coli. Zeitschrift für Naturforsch. C 54:1055-1067. doi 10.1515/znc-1999-1210
Dong Q, Fromm H J. (1990) Chemical modification of adenylosuccinate synthetase from Escherichia coli by pyridoxal 5′-phosphate. Identification of an active site lysyl residue. J Biol Chem. 265 (11): 6235-40. doi: lack
Ducati R G, Namanja-Magliano H A, Schramm V L (2013) Transition-state inhibitors of purine salvage and other prospective enzyme targets in malaria Future Med Chem. 5 (11): 1341-1360. doi: 10.4155/fmc.13.51
Dziekan J M, Yu H, Chen D, Dai L, Wirjanata G, Larsson A, Prabhu N, Sobota R M, Bozdech Z, Pär Nordlund P. (2019) Identifying purine nucleoside phosphorylase as the target of quinine using cellular thermal shift assay Sci Transl Med 11 (473): eaau3174. doi: 10.1126/scitranslmed.aau3174
Emsley P, Lohkamp B, Scott W G, Cowtan K. Features and development of Coot (2020) Acta Cryst D 66:486-501. doi: 10.1107/S0907444910007493
Dzierżanowska-Fangrat K, Rożynek E, Celińska-Cedro D et al. (2005) Antimicrobial resistance of Helicobacter pylori in Poland: a multicenter study. Int. J. Antimicrob. Agents 26:230-234. doi: 10.1016/j.ijantimicag.2005.06.015
EUCAST (2021) Breakpoint tables for interpretation of MICs and zone diameters, Version 11.0, 2021. http://www.eucast.org
Ferrero R L, Cussac V, Courcoux P, Labigne A. (1992) Construction of isogenic urease-negative mutants of Helicobacter pylori by allelic exchange. J Bacteriol. 174 (13): 4212-4217. doi: 10.1128/jb.174.13.4212-4217.1992
Go M F (2002) Review article: natural history and epidemiology of Helicobacter pylori infection. Aliment Pharmacol Ther. 10, 3-15. doi: 10.1046/j.1365-2036.2002.0160s1003.x
Hanessian S, Lu P P, Sanceau J Y, Chemla P, Gohda K, Fonne-Pfister R, Prade L, Cowan-Jacob S W (1999) An enzyme-bound bisubstrate hybrid inhibitor of adenylosuccinate synthetase. Angew Chem Int Ed Engl 38:3159-3162. doi: 10.1007/s00253-021-11510-9
Huang C C, Tsai K W, Tsai T J, Hsu P I (2017) Update on the first-line treatment for Helicobacter pylori infection—a continuing challenge from an old enemy. Biomark. Res. 5:23. doi: 10.1186/s40364-017-0103-x
Irie Y, Tateda K, Matsumoto T, Miyazaki S, Yamaguchi K (1997) Antibiotic MICs and short time-killing against Helicobacter pylori: therapeutic potential of kanamycin. J Antimicrob Chemother. 40 (2): 235-40. doi: 10.1093/jac/40.2.235
Jones J W, Robins R K (1963) Purine Nucleosides. III. Methylation Studies of Certain Naturally Ocurring Purine Nucleosides, J. Am. Chem. Soc. 85:193-201. doi: 10.1021/ja00885a019
Kabsch W (2010) Xds. Acta Crystallogr. D. Biol. Crystallogr. 66, 125-32. doi: 10.1107/S0907444909047337
Knezevic P, Aleksic Sabo V, Simin N, Lesjak M, Mimica-Dukic N. (2018) A colorimetric broth microdilution method for assessment of Helicobacter pylori sensitivity to antimicrobial agents. J Pharm Biomed Anal. 152:271-278. doi: 10.1016/j.jpba.2018.02.003
Krzyżek P, Franiczek R, Krzyzanowska B, Łaczmanski Ł, Migdał P, Gosciniak G (2019) In vitro activity of sertraline, an antidepressant, against antibiotic-susceptible and antibiotic-resistant Helicobacter pylori strains, Pathogens 8:228. doi: 10.3390/pathogens8040228
Kulikowska E, Bzowska A, Wierzchowski J, Shugar D (1986) Properties of two unusual and fluorescent substrates of purine nucleoside phosphorylase: 7-methylguanosine and 7-methylinosine. Biochim. Biophys. Acta 874:355-363. doi: 10.1016/0167-4838 (86) 90035-x
Liebschner D, Afonine P V, Baker M L, Bunkóczi G, Chen V B, Croll T I, Hintze B, Hung L-W, Jain S, McCoy A J, Moriarty N W, Oeffner R D, Poon B K, Prisant M G, Read R J, Richardson J S, Richardson D C, Sammito M D, Sobolev O V, Stockwell D H, Terwilliger T C, Urzhumtsev A G, Videau L L, Williams C J, Adams P D (2019) Macromolecular Structure Determination Using X-Rays, Neutrons and Electrons: Recent Developments in Phenix. Acta Crystallogr. Sect. D Struct. Biol. 75 (10): 861-877. doi: 10.1107/S2059798319011471
Liechti G, Goldberg J B (2012) Helicobacter Pylori Relies Primarily on the Purine Salvage Pathway for Purine Nucleotide Biosynthesis. J. Bacteriol. 194:839-54. doi: 10.1128/J B.05757-11
Makola D, Peura D A, Crowe S E (2007) Helicobacter pylori infection and related gastrointestinal diseases. J. Clin. Gastroenterol. 41:548-558. doi: 10.1097/MCG.0b013e318030e3c3
Malfertheiner P, Selgrad M, Bornschein J, Helicobacter pylori: clinical management. Curr. Opin Gastroenterol. 28, 608-614 (2012). doi: 10.1097/MOG.0b013e32835918a7
de Martel C, Llosa A E, Friedman G D, Vogelman J H, Orentreich N, Stolzenberg-Solomon R Z, Personnet J, Helicobacter pylori infection and development of pancreatic cancer. Cancer Epidemiol Biomarkers Prev 17,1188-1194 (2008). doi: 10.1158/1055-9965.EPI-08-0185
McCoy A.J, Grosse-Kunstleve R W, Adams P D, Winn M D, Storoni L C, Read R J, Phaser crystallographic software. J Appl Cryst 40, 658-674 (2007). doi: 10.1107/S0021889807021206
Murshudov G N, Skubák P, Lebedev A A, Pannu N S, Steiner R A, Nichills R A, Winn M D, Long F, Vagin A A (2011) REFMAC5 for the refinement of macromolecular crystal structures. Acta Cryst D 67:355-367. doi: 10.1107/S0907444911001314
Narczyk M, Bertoša B, Papa L, Vuković V, Leščić Ašler I, Wielgus-Kutrowska B, Bzowska A, Luić M, Štefanić Z (2018) Helicobacter pylori purine nucleoside phosphorylase shows new distribution patterns of open and closed active site conformations and unusual biochemical features. FEBS J 285:1305-1325. doi: 10.1111/febs. 14403
Pillai S K, Eliopoulos G M, Moellering R C (2005) Antimicrobial combination. V. Lorian (Ed.), Antibiotics in Laboratory, Lippincott, Williams & Wilkins, Philadelphia, pp. 365-409
Raj D S, Kesavan D K, Muthusamy N (2021) Efflux pumps potential drug targets to circumvent drug Resistance-Multi drug efflux pumps of Helicobacter pylori, Materials Today: Proceedings 45 (4), doi: 10.1016/j.matpr.2020.11.955
Rudolph F B, Fromm H J. Initial rate studies of adenylosuccinate synthetase with product and competitive inhibitors. J Biol Chem 1969; 244:3832-9. https://doi.org/10.1016/S0021-9258 (17) 36425-6
Ruggiero P, Helicobacter pylori infection: what's new. Curr. Opin. Infect, Dis. 25, 337-344 (2012). doi: 10.1097/QCO.0b013e3283531f7c.
Schmitt W, Haas R (1994) Genetic analysis of the Helicobacter pylori vacuolating cytotoxin: structural similarities with the IgA protease type of exported protein. Mol. Microbiol. 12:307-319. doi: 10.1111/j.1365-2958.1994.tb01019.x
Schramm V L (2018) Enzymatic Transition States and Drug Design. Chem Rev. 118:11194-11258. doi: 10.1021/acs.chemrev.8b00369
Segel I H (1993) Enzyme kinetics: Behavior and Analysis of Rapid Equilibrium and Steady-State Enzyme Systems. (Wiley Classic Library, eds), John Wiley & sons, New York.
Štefanić Z, Mikleušević G, Luić M, Bzowska A, Leščić Ašler I (2017) Structural characterization of purine nucleoside phosphorylase from human pathogen Helicobacter pylori. Int J Biol Macromol 101:518-526. doi: 10.1016/j.ijbiomac.2017.03.101
Tomb J F, White O, Kerlavage A R, Clayton R A, Sutton G G, Fleischmann R D, Ketchum K A, Klenk H P, Gill S, Dougherty B A, Nelson K, Quackenbush J, Zhou L, Kirkness E F, Peterson S, Loftus B, Richardson D, Dodson R, Khalak H G, Glodek A, McKenney K, Fitzegerald L M, Lee N, Adams M D, Hickey E K, Berg D E, Gocayne J D, Utterback T R, Peterson J D, Kelley J M, Cotton M D, Weldman J M, Fujii C, Bowman C, Watthey L, Wallin E, Hayes W S, Borodovsky M, Karp P D, Smith H O, Fraser C M, Venter J C (1997) Erratum: The complete genome sequence of the gastric pathogen Helicobacter pylori (Nature (1997) 388 (539-547)). Nature 389:412. doi: 10.1038/38792
Vagin A, Teplyakov A (1997) MOLREP: an Automated Program for Molecular Replacement. J. Appl. Crystallogr. 30, 1022-1025. doi: 10.1107/S0907444909042589
Walters E W, Lee S-F, Niderman T, Bernasconi P, Subramanian M V, Siehl D L (1997) Adenylosuccinate Synthetase from Maize. Purification, Properties, and Mechanism of Inhibition by 5′-Phosphohydantocidin. Plant Physiol. 114:549-555. doi: 10.1104/pp. 114.2.549
Wojtyś M, Jaźwiec R, Kazazić S, Leščić Ašler L, Knežević P, Aleksić Sabo V, Luić M, Jagusztyn-Krynicka E K, Bzowska A (2021) A comprehensive method for determining cellular uptake of purine nucleoside phosphorylase and adenylosuccinate synthetase inhibitors by H. pylori, Appl Microbiol Biotechnol. 105:7949-7967. doi: 10.1007/s00253-021-11510-9.

1. A method of treating an H. pylori infection and/or treating or preventing a disease associated with an H. pylori infection, the method comprising administering to an individual a pharmaceutically effective amount of an inhibitor of the Helicobacter pylori (H. pylori) cell purine nucleotide salvage pathway (reserve) enzyme.

2. The method of claim 1, characterised in that the inhibitor is an inhibitor of the enzyme selected from purine nucleoside phosphorylase (PNP) and adenylosuccinate synthetase (AdSS), or is a metabolic precursor of such an inhibitor.

3. The method of claim 1, characterised in that the inhibitor used is an PNP enzyme inhibitor.

4. The method of claim 3, characterised in that the inhibitor is selected from substituted purines (Formula I), substituted 9-deaza-purines (Formula II), substituted 8-aza-9-deaza-purines (Formula III), mefloquine and quinine.

5. The method of claim 4, characterised in that the inhibitor is:
(a) 6-methylformycin A;
(b) immucillin, preferably immucillin A (Formula IIa);
(c) a substituted purine selected from 6-benzyloxy-2-chloropurine (6BnO-2Cl-Pu, Formula IV), 6-benzylthio-2-chloropurine (6BnS-2Cl-Pu, Formula V), 2-chloro-6-benzylthiopurine-2′-deoxy-9-ribofuranoside (6BnS-2Cl-Pu-9dr, Formula VI), 6-benzylthiopurine (6BnS-Pu, Formula VII) and 2, 6-dichloropurine (2, 6-diCl-Pu, Formula VIII), preferably 6BnS-2Cl-Pu (Formula V), more preferably 6BnS-2Cl-Pu (Formula V) in crystalline form; or
(d) mefloquine or quinine.

6-11. (canceled)

12. The method of claim 1, characterised in that the inhibitor is an AdSS enzyme inhibitor or a metabolic precursor thereof.

13. The method of claim 12, characterised in that the inhibitor is selected from hydantocidin, hydantocidin 5′-phosphate, hybrid of hadacidin and hydantocidin 5′-phosphate, pyridoxal 5′-phosphate (vitamin B6) and a metabolic precursor of pyridoxal 5′-phosphate, preferably pyridoxal.

14. The method of claim 13, characterised in that the inhibitor is pyridoxal 5′-phosphate (vitamin B6) or its metabolic precursor, preferably pyridoxal.

15. The method of claim 1, comprising administering both a PNP inhibitor and an Adss inhibitor.

16. The method of claim 1, characterised in that the disease associated with H. pylori infection is a disease selected from the group consisting of gastritis, duodenitis, gastric ulcer, duodenal ulcer, gastric cancer, MALT-type lymphoma, pancreatic cancer and neurodegenerative disorder.

17. The method of claim 1, characterised in that the H. pylori belongs to an antibiotic-resistant strain, preferably wherein the H. pylori strain is resistant to clarithromycin and/or metronidazole.

18. (canceled)

19. The method of claim 17, characterised in that the inhibitor of the enzyme used is:
(a) a PNP enzyme inhibitor selected from quinine or mefloquine; or
(b) an AdSS enzyme inhibitor selected from pyridoxal 5′-phosphate (PLP) and its precursor, i.e. pyridoxal, preferably PI-h.

20. (canceled)

21. The method of claim 1, comprising administering the inhibitor of the Helicobacter pylori (H. pylori) cell purine nucleotide salvage pathway (reserve) enzyme in combination with:
(a) at least one antibiotic, selected in particular from the group consisting of metronidazole, clarithromycin, tetracycline, amoxicillin, levofloxacin, sitafloxacin and rifabutin, especially with at least two antibiotics from this group;
(b) a proton pump inhibitor (PPI), selected in particular from the group consisting of omeprazole, lansoprazole, esomeprazole, rabeprazole and pantoprazole;
(c) bismuth salt, in particular with bismuth citrate;
(d) an antibiotic, wherein preferably the PNP enzyme inhibitor is 6BnS-2Cl-Pu and the antibiotic is metronidazole.

22-24. (canceled)

25. The method of claim 1, wherein the inhibitor of the Helicobacter pylori (H. pylori) cell purine nucleotide salvage pathway (reserve) enzyme is administered in combination with an antibiotic, wherein preferably the PNP enzyme inhibitor is 6BnS-2Cl-Pu and the antibiotic is metronidazole, characterised in that it is additionally administered in combination with an additional antibiotic and/or proton pump inhibitor and/or bismuth salt.

26. The method of claim 1, comprising administering a pharmaceutical composition comprising the inhibitor of the H. pylori cell purine nucleotide salvage pathway and at least one pharmaceutically acceptable excipient.

27. The method of claim 26, characterised in that the pharmaceutical composition comprises a PNP enzyme inhibitor, in particular 6BnS-2Cl-Pu, immucillin A, or quinine or mefloquine.

28. The method of claim 26, characterised in that the pharmaceutical composition comprises an AdSS enzyme inhibitor, in particular pyridoxal 5′-phosphate (vitamin B6) or its metabolic precursor, preferably pyridoxal.

29. The method of claim 26, wherein the pharmaceutical composition further comprises at least one additional drug for the treatment of H. pylori infection, selected in particular from an antibiotic, a proton pump inhibitor and a bismuth salt.

30. The method of claim 29, characterised in that the pharmaceutical composition comprises a PNP enzyme inhibitor, in particular 6BnS-2Cl-Pu, immucillin A, or quinine or mefloquine, and at least one antibiotic, in particular metronidazole.

31. The method of claim 30, characterised in that the pharmaceutical composition additionally comprises a proton pump inhibitor and/or a bismuth salt.
